SEQUENCE LISTING (44 sequences)

SEQ ID NO: 1 - DNA, 26 bp, Artificial Sequence
  Description of Artificial Sequence Synthetic primer
tgaaggtcggagtcaacggatttggt

SEQ ID NO: 2 - DNA, 24 bp, Artificial Sequence
  Description of Artificial Sequence Synthetic primer
catgtgggccatgaggtccaccac

SEQ ID NO: 3 - DNA, 20 bp, Artificial Sequence
  Description of Artificial Sequence Synthetic primer
ctcttccagccttccttcct

SEQ ID NO: 4 - DNA, 20 bp, Artificial Sequence
  Description of Artificial Sequence Synthetic primer
tcgtcatactcctgcttgct

SEQ ID NO: 5 - DNA, 24 bp, Artificial Sequence
  Description of Artificial Sequence Synthetic primer
cagccatgtacgttgctatccagg

SEQ ID NO: 6 - DNA, 23 bp, Artificial Sequence
  Description of Artificial Sequence Synthetic primer
aggtccagacgcaggatggcatg

SEQ ID NO: 7 - DNA, 20 bp, Artificial Sequence
  Description of Artificial Sequence Synthetic primer
attcagccaaacgaccatct

SEQ ID NO: 8 - DNA, 20 bp, Artificial Sequence
  Description of Artificial Sequence Synthetic primer
cagcagcctcaaaatcctct

SEQ ID NO: 9 - DNA, 23 bp, Artificial Sequence
  Description of Artificial Sequence Synthetic primer
acacctggcttcggatttcgcct

SEQ ID NO: 10 - DNA, 24 bp, Artificial Sequence
  Description of Artificial Sequence Synthetic primer
gcttcctccacccacttctgcagc

SEQ ID NO: 11 - DNA, 18 bp, Artificial Sequence
  Description of Artificial Sequence Synthetic primer
cccccggcggcaatagca

SEQ ID NO: 12 - DNA, 20 bp, Artificial Sequence
  Description of Artificial Sequence Synthetic primer
tcggcgccggggagatacat

SEQ ID NO: 13 - DNA, 20 bp, Artificial Sequence
  Description of Artificial Sequence Synthetic primer
taccctctcaacgacagcag

SEQ ID NO: 14 - DNA, 20 bp, Artificial Sequence
  Description of Artificial Sequence Synthetic primer
tcttgacattctcctcggtg

SEQ ID NO: 15 - DNA, 20 bp, Artificial Sequence
  Description of Artificial Sequence Synthetic primer
tctcctcttccctcctccat

SEQ ID NO: 16 - DNA, 20 bp, Artificial Sequence
  Description of Artificial Sequence Synthetic primer
ggatgttctgggtctggttg

SEQ ID NO: 17 - DNA, 20 bp, Artificial Sequence
  Description of Artificial Sequence Synthetic primer
gagagagaccgaggagttca

SEQ ID NO: 18 - DNA, 20 bp, Artificial Sequence
  Description of Artificial Sequence Synthetic primer
cctttgctgacgctgatgac

SEQ ID NO: 19 - DNA, 16 bp, Artificial Sequence
  Description of Artificial Sequence Synthetic primer
cagcgacgcgctgctc

SEQ ID NO: 20 - DNA, 20 bp, Artificial Sequence
  Description of Artificial Sequence Synthetic primer
tgcaggaaccgggtggcatg

SEQ ID NO: 21 - DNA, 21 bp, Artificial Sequence
  Description of Artificial Sequence Synthetic primer
ggtgaccaagttcatgctgtg

SEQ ID NO: 22 - DNA, 20 bp, Artificial Sequence
  Description of Artificial Sequence Synthetic primer
gtgtccttgatccacttccg

SEQ ID NO: 23 - DNA, 20 bp, Artificial Sequence
  Description of Artificial Sequence Synthetic primer
gaagccattgagccaggtgt

SEQ ID NO: 24 - DNA, 20 bp, Artificial Sequence
  Description of Artificial Sequence Synthetic primer
tcgtccacgtgtaagttgcg

SEQ ID NO: 25 - DNA, 21 bp, Artificial Sequence
  Description of Artificial Sequence Synthetic primer
actggcagcaacagtcttacc

SEQ ID NO: 26 - DNA, 20 bp, Artificial Sequence
  Description of Artificial Sequence Synthetic primer
tcgtccacgtgtaagttgcg

SEQ ID NO: 27 - DNA, 21 bp, Artificial Sequence
  Description of Artificial Sequence Synthetic primer
gaacgcattgccacatacact

SEQ ID NO: 28 - DNA, 21 bp, Artificial Sequence
  Description of Artificial Sequence Synthetic primer
ctgtggaggtggtgagagaga

SEQ ID NO: 29 - DNA, 20 bp, Artificial Sequence
  Description of Artificial Sequence Synthetic primer
gcacggcttttgttcagatg

SEQ ID NO: 30 - DNA, 20 bp, Artificial Sequence
  Description of Artificial Sequence Synthetic primer
gttcttggctttcaggatgg

SEQ ID NO: 31 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 32 - DNA, 1083 bp, Homo sapiens
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 33 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 34 - DNA, 1155 bp, Homo sapiens
catgagtcagtgaacagggaatgggtgaatgacatttgtgggtaggttatttctagaagttaggtgggcagcttggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 35 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 36 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 37 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 38 - DNA, 954 bp, Homo sapiens
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 39 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 40 - DNA, 1365 bp, Homo sapiens
ctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 41 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 42 - DNA, 1413 bp, Homo sapiens
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 43 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 44 - DNA, 4815 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

(19) United States
(12) Patent Application Publication
(10) Pub. No.: US 2011/0171221 A1
(43) Pub. Date: Jul. 14, 2011

(54) COMPOSITIONS AND METHODS FOR THE TREATMENT OF A NEOPLASIA

(75) Inventors: Johannes Vieweg, Gainesville, FL (US); Zhen Su, Toronto (CA); Dennis A. Steindler, Gainesville, FL (US)

(73) Assignee: University of Florida Research Foundation, Gainesville, FL (US)

(21) Appl. No.: 12/901,153

(22) Filed: Oct. 8, 2010

Related U.S. Application Data
(63) Continuation of application No. PCT/US2009/002262, filed on Apr. 10, 2009.
Application No. PCT/US2009/002262 is a continuation-in-part of application No. PCT/US2008/059966, filed on Apr. 10, 2008.
(60) Provisional application No. 61/123,867, filed on Apr. 10, 2008.

(51) Int. Cl. A61K 39/395; C12N 5/09; A61K 31/7088; A61K 31/713; C12Q 1/68; G01N 33/574; C12N 5/071; C07K 16/32; C07H 21/00; A61P 35/00

(52) U.S. Cl. 424/138.1; 435/375; 514/44 R; 514/44 A; 435/6.14; 436/501; 435/6.11; 435/6.13; 435/7.1; 435/325; 530/387.7; 536/24.5; 536/24.33

(57) ABSTRACT

Compositions and methods for the diagnosis, treatment and prevention of cancer, including prostate cancer and renal cell carcinoma, as well as for treatment selection.

### CROSS-REFERENCE TO RELATED APPLICATION

This application claims the benefit of the following U.S. Provisional Application Ser. No. 61/123,867, filed Apr. 10, 2008; the entire contents of which are incorporated herein by this reference.

### STATEMENT OF RIGHTS TO INVENTIONS MADE UNDER FEDERALLY SPONSORED RESEARCH

This work was supported by the following grant from the National Institutes of Health, Grant No: HL-70143. The government has certain rights in the invention.

### BACKGROUND OF THE INVENTION

Cancer is a leading healthcare concern in North America and Europe. There were an estimated 232,090 new cases of prostate cancer diagnosed in 2005 in the United States, and over 30,350 deaths from advanced metastatic disease. Prostate cancer is now the most commonly diagnosed lethal malignancy, and the second leading cause of cancer death of men in the United States. Although curative treatment (e.g., radical prostatectomy or radiotherapy) is feasible for many patients with the earliest stage disease, a subset of patients have prostate cancer that is resistant to conventional treatments, that is locally advanced, or that is metastatic. Metastatic prostate cancer is initially treated with androgen deprivation, which achieves stabilization or regression of disease in more than 80% of patients. Nevertheless, all patients with metastatic prostate cancer ultimately develop androgen resistant disease. The median survival for such patients is approximately one year. Treatment recommendations for subjects with metastatic prostate cancers include experimental therapy conducted in the setting of peer reviewed clinical trials, underscoring the fact that current standard therapies are inadequate and new approaches of treatment are needed.

### SUMMARY OF THE INVENTION

As described below, the present invention features compositions and methods for the diagnosis, treatment and prevention of neoplasia, as well as for treatment selection.

In one aspect, the invention provides method for reducing the survival of a neoplastic cell (neoplasia, prostate cancer, bladder cancer, renal carcinoma, breast cancer, melanoma,

In another aspect, the invention provides a method of inducing cell death (e.g., apoptotic or necrotic) in a neoplastic cell, the method comprising contacting the cell with an agent that reduces the expression of a OCT3/4, Nanog, Sox2, c-Myc or Klf4 nucleic acid molecule or polypeptide. In one embodiment, the agent is an inhibitory nucleic acid molecule (e.g., an antisense molecule, shRNA, or siRNA) at least a portion of which is complementary to an OCT3/4, Nanog, Sox2, c-Myc or Klf4 nucleic acid molecule. In another embodiment, the agent is an antibody that specifically binds a OCT3/4, Nanog, Sox2, c-Myc or Klf4 polypeptide. In another embodiment, the method reduces the expression of any two, three, four, or five of OCT3/4, Nanog, Sox2, c-Myc or Klf4 nucleic acid molecules or polypeptides.

In another aspect, the invention provides a method for treating or preventing neoplasia in a subject in need thereof, the method comprising contacting a neoplasia cell in the subject with an agent that reduces the expression of a OCT3/4, Nanog, Sox2, c-Myc and Klf4 nucleic acid molecule or polypeptide. In one embodiment, the agent is an inhibitory nucleic acid molecule at least a portion of which is complementary to an OCT3/4, Nanog, Sox2, c-Myc or Klf4 nucleic acid molecule. In another embodiment, the inhibitory nucleic acid molecule is an antisense molecule, shRNA, or siRNA. In another embodiment, the agent is an antibody that specifically binds a OCT3/4, Nanog, Sox2, c-Myc or Klf4 polypeptide.

In another aspect, the invention provides a method for identifying a neoplasia in a subject, the method comprising identifying an increased level of a nucleic acid molecule or polypeptide Marker selected from any one or more of OCT3/4, Nanog, Sox2, c-Myc and Klf4 in a biological sample derived from the subject, relative to the level present in a reference, thereby identifying the subject as having neoplasia.

In yet another aspect, the invention provides a method for identifying a metastatic neoplasia or a neoplasia having a propensity to metastasize, the method comprising comparing the level of a nucleic acid molecule or polypeptide Marker selected from any one or more of OCT3/4, Nanog, Sox2, c-Myc and Klf4 in a biological sample, relative to the level present in a reference, wherein an increase in the level of one or more of said Markers identifies the neoplasia as metastatic or as having a propensity to metastasize. In one embodiment, the absence of an increase in the level of one or more Markers identifies the neoplasia as non-metastatic or as lacking the propensity to metastasize.

In yet another aspect, the invention provides a method for identifying a subject as having or having a propensity to develop a metastatic prostate carcinoma, the method comprising comparing the level of a nucleic acid molecule or polypeptide Marker selected from any one or more of OCT3/4, Nanog, Sox2, c-Myc or Klf4 in a biological sample derived from the subject relative to the level present in a reference, wherein an increase in the level of one or more of said Markers identifies the neoplasia as metastatic or as having a propensity to metastasize. In one embodiment, the absence of an increase in the level of one or more Markers identifies the neoplasia as non-metastatic or as lacking the propensity to develop a metastatic carcinoma.

In still another aspect, the invention provides a method of determining the prognosis of a subject having neoplasia, the method comprising determining the level of a nucleic acid molecule or polypeptide Marker selected from any one or more of OCT3/4, Nanog, Sox2, c-Myc and Klf4 in a biological sample derived from the subject, relative to the level present in a reference. In one embodiment, an increase in the level of each of said Markers identifies the subject as having a poor prognosis. In another embodiment, the absence of alteration in the level of one or more of said Markers identifies the subject as having a good prognosis.

In another aspect, the invention provides a method of selecting an appropriate therapy for a subject having neoplasia, the method comprising comparing the level of a Marker selected from any one or more of OCT3/4, Nanog, Sox2, c-Myc and Klf4 nucleic acid molecule or polypeptide in a biological sample derived from the subject, relative to the level present in a reference, wherein the an increase in the level of all of said Markers indicates that aggressive therapy is appropriate for the subject, and the absence of an increase in the level of all of said Markers indicates that conventional therapy is appropriate.

In another aspect, the invention provides a method of monitoring neoplasia therapy in a subject, the method comprising determining the level of a Marker selected from any one or more of OCT3/4, Nanog, Sox2, c-Myc and Klf4 nucleic acid molecule or polypeptide in a biological sample derived from the subject, relative to the level present in a reference, wherein a reduction in the level of said marker.

In another aspect, the invention provides a method of identifying a neoplasia as resistant to treatment with a conventional therapy, the method comprising identifying an increased level of a Marker selected from any one or more of OCT3/4, Nanog, Sox2, c-Myc and Klf4 nucleic acid molecule or polypeptide in a biological sample derived from the subject, relative to the level present in a reference, wherein the increased level of said Markers identifies the neoplasia as resistant to treatment with a conventional therapy.

In another aspect, the invention provides an isolated stem-like neoplasia-initiating cell having increased expression of two, three, four or five of an OCT3/4, Nanog, Sox2, c-Myc and Klf4 nucleic acid molecule or polypeptide.

In still another aspect, the invention provides a method of identifying an agent that induces cell death in an isolated stem-like neoplasia-initiating cell having increased expression of two or more of an OCT3/4, Nanog, Sox2, c-Myc and Klf4 nucleic acid molecule or polypeptide, the method comprising contacting the cell with a test agent and assaying for an increase in cell death, thereby identifying the agent as inducing cell death.

In another aspect, the invention provides a method of identifying an agent for treating or preventing neoplasia or metastatic disease, the method comprising contacting the cell with a test agent and assaying for reduction in cell proliferation or survival, thereby identifying the agent as treating or preventing neoplasia or metastatic disease.

In another aspect, the invention provides a pharmaceutical composition for the treatment or prevention of neoplasia, the composition comprising an agent that reduces the expression or activity of a polypeptide or nucleic acid molecule selected from any one or more of OCT3/4, Nanog, Sox2, c-Myc and Klf4 nucleic acid molecule or polypeptide. In one embodiment, the agent is an inhibitory nucleic acid molecule selected from any one or more of OCT3/4, Nanog, Sox2, c-Myc and Klf4.

In another aspect, the invention provides a method of selecting a treatment for a subject diagnosed as having neoplasia, the method involving quantifying the level of OCT3/4, Nanog, Sox2, c-Myc or Klf4 in a biologic sample from the subject relative to a reference, wherein the presence or level of expression of OCT3/4, Nanog, Sox2, c-Myc or Klf4 is indicative of a treatment; and selecting a treatment.

In another aspect, the invention provides a method of selecting a treatment for a subject diagnosed as having neoplasia, the method involving quantifying the level of OCT3/4, Nanog, Sox2, c-Myc or Klf4 in a subject sample; and selecting a treatment for the subject, wherein the treatment is selected from any one or more of surveillance, surgery, hormone therapy, chemotherapy, and radiotherapy.

In another aspect, the invention provides a method for determining the Marker profile of a neoplasia, the method comprising quantifying the level of two or more Markers selected from any one or more of OCT3/4, Nanog, Sox2, c-Myc or Klf4 in a biologic sample, wherein the level of Marker in the sample relative to the level in a reference determines the Marker profile of the prostatic neoplasia.

In another aspect, the invention provides a kit for the analysis of OCT3/4, Nanog, Sox2, c-Myc or Klf4, the kit comprising at least one primer or antibody capable of specifically binding or hybridizing to OCT3/4, Nanog, Sox2, c-Myc or Klf4 polypeptide or nucleic acid molecule, and directions for using the primer or antibody for the analysis of OCT3/4, Nanog, Sox2, c-Myc or Klf4. In one embodiment, antibody binding is detected by fluorescence, by autoradiography, by an immunoassay, by an enzymatic assay, or by a colorimetric assay.

In another aspect, the invention provides a microarray comprising at least two (e.g., 2, 3, 4, or 5) nucleic acid molecules, or fragments thereof, bound to a solid support, wherein the two nucleic acid molecules are any one or more of OCT3/4, Nanog, Sox2, c-Myc or Klf4.

In another aspect, the invention provides an isolated population of stem-like neoplasia-initiating cells having increased expression of two, three, four or more of an OCT3/4, Nanog, Sox2, c-Myc and Klf4 nucleic acid molecule or polypeptide.

In another aspect, the invention provides a method of isolating a stem-like cancer-initiating cell, involving selecting a cell having increased expression of two or more of an OCT3/4, Nanog, Sox2, c-Myc and Klf4 nucleic acid molecule or polypeptide, to thereby isolate a stem-like cancer-initiating cell. In one embodiment, the method further involves selecting a cell for the expression of E-cadherin prior to selecting for increased expression of two, three, four or more of an OCT3/4, Nanog, Sox2, c-Myc and Klf4 nucleic acid molecule or polypeptide.

In various embodiments of the invention, the cells or tissues are derived from any one or more of prostate tissue, renal tissue, bladder tissue, breast tissue, skin, and connective tissue. In other various embodiments of the above aspects, the neoplasia is any one or more of prostate cancer, renal carcinoma, bladder cancer, breast cancer, melanoma, and sarcoma. In various embodiments of the above aspects, the biological sample is a biologic fluid (e.g., blood, serum, plasma, ejaculate, or urine). In other embodiments, the reference is the level of OCT3/4, Nanog, Sox2, c-Myc or Klf4 polypeptide or nucleic acid molecule present in a control sample, e.g., a control sample derived from a healthy subject or a subject with a non-metastatic neoplasia or a control sample derived from the same subject at an earlier point in time. In still other embodiments of the above aspects, the method reduces or measures the expression of any two, three, four, or five of OCT3/4, Nanog, Sox2, c-Myc or Klf4 nucleic acid molecules or polypeptides.

The invention provides compositions and methods for diagnosing, treating or preventing cancer. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. By “OCT3/4 polypeptide” is meant a polypeptide or fragment thereof having at least 85% amino acid identity to NCBI Accession No. NP_{—}002692 and having DNA binding activity.

By “OCT3/4 nucleic acid molecule” is meant a polynucleotide encoding an OCT3/4 polypeptide. An exemplary OCT3/4 nucleic acid molecule is provided at NCBI Accession No. NM_{—}203289.

By “NANOG polypeptide” is meant a polypeptide or fragment thereof having at least 85% amino acid identity to NCBI Accession No. NP_{—}079141.2 and having DNA binding activity.

By “NANOG nucleic acid molecule” is meant a polynucleotide encoding a NANOG polypeptide. An exemplary NANOG nucleic acid molecule is provided at NCBI Accession No. NM_{—}024865.2.

By “SOX2 polypeptide” is meant a polypeptide or fragment thereof having at least 85% amino acid identity to NCBI Accession No. NP_{—}003097 and having DNA binding activity.

By “SOX2 nucleic acid molecule” is meant a polynucleotide encoding a SOX2 polypeptide. An exemplary SOX2 nucleic acid molecule sequence is provided at NCBI Accession No. NM_{—}003106.

By “C-MYC polypeptide” is meant a polypeptide or fragment thereof having at least 85% amino acid identity to NCBI Accession No. NP_{—}002458. By “C-MYC nucleic acid molecule” is meant a polynucleotide encoding a C-MYC polypeptide. An exemplary C-MYC nucleic acid molecule sequence is provided at NCBI Accession No. NM_{—}002467.

By “KLF4 polypeptide” is meant a polypeptide or fragment thereof having at least 85% amino acid identity to NCBI Accession No. NP_{—}004226 and having DNA binding activity.

By “KLF4 nucleic acid molecule” is meant a polynucleotide encoding a KLF4 polypeptide. An exemplary KLF4 nucleic acid molecule sequence is provided at NCBI Accession No. NM_{—}004235.

By “E-cadherin polypeptide” is meant a polypeptide or fragment thereof having at least 85% amino acid identity to NCBI Accession No. CAA78353 and having cell surface expression.

By “E-cadherin nucleic acid molecule” is meant a polynucleotide encoding an E-cadherin polypeptide. An exemplary E-cadherin nucleic acid molecule sequence is provided at NCBI Accession No. NM_{—}004360.

Select exemplary sequences delineated herein are shown at FIG. 19.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

By “biologic sample” is meant any tissue, cell, fluid, or other material derived from an organism.

By “cancer stem cell” or “stem-like cancer-initiating cell” is meant cells that can neoplastic and can undergo self-renewal as well as abnormal proliferation and differentiation. Functional features of cancer stem cells are that they are tumorigenic; they can give rise to additional neoplastic cells by self-renewal; and they can give rise to non-tumorigenic neoplastic cells. Without being bound to any particular theory, cancer stem cells contribute to the development of metastatic cancer.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

By “neoplasia” is meant any disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “reference” is meant a standard of comparison. For example, the OCT3/4, NANOG, SOX2, C-MYC or KLF4 polypeptide or polynucleotide level present in a patient sample may be compared to the level of said polypeptide or polynucleotide present in a corresponding healthy cell or tissue or in a neoplastic cell or tissue that lacks a propensity to metastasize.

By “periodic” is meant at regular intervals. Periodic patient monitoring includes, for example, a schedule of tests that are administered daily, bi-weekly, bi-monthly, monthly, bi-annually, or annually.

By “severity of neoplasia” is meant the degree of pathology. The severity of a neoplasia increases, for example, as the stage or grade of the neoplasia increases.

By “Marker profile” is meant a characterization of the expression or expression level of two or more polypeptides or polynucleotides.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1C show the identification of stem cells from metastatic prostate cancer (PCa) cell lines. FIG. 1A shows the results of RT-PCR analysis for the detection of mRNA levels of CD133, Nanog and OCT3/4 in DU145, LNCaP and PC3 prostate cancer cell lines. Data were normalized to β-actin expression. Representative results of three independent experiments are shown. FIG. 1B shows Western blot analysis for the detection of protein levels of Nanog and OCT3/4 in DU145, LNCaP and PC3 PC cell lines. Data were normalized to β-actin expression. Representative results of three independent experiments are shown. FIG. 1C shows DU145, LNCaP and PC3 cells immunostained for OCT3/4 (red), Nanog (green) and the merge of OCT3/4 and Nanog (OCT3/4+Nanog, yellow). Phase contrast images served as controls. Representative results of three independent experiments are shown.

FIGS. 2A-2C show the identification of stem cell-like tumor cells with pluripotent stem cell reprogramming factors in prostate cancer cell lines. FIG. 2A shows the results of RT-PCR analysis for detecting expression levels of OCT3/4, SOX2, Nanog, c-Myc and Klf4 in DU145 and PC3 cell lines, with human Embryonic Stem Cells (hESC) as the control. Data were normalized to β-actin expression. Representative results of three independent experiments are shown. FIG. 2B shows the results of Western blot analysis for detecting expression levels of OCT3/4, SOX2, Nanog, c-Myc and Klf4 in DU145 and PC3 cell lines, with human Embryonic Stem Cells (hESC) as the control. Data were normalized to β-actin expression. Representative results of three independent experiments are shown. FIG. 2C depicts images of DU145 and PC3 cells immunostained for OCT3/4 (red), SOX2 (green), DAPI (blue); OCT3/4 and SOX2 staining were also merged. Magnification is 40× and the scale bar represents 20 μm. Representative results of three independent experiments are shown.

FIGS. 3A-3D show that prostate cancer stem cells express E-cadherin and can be sorted from non-stem prostate cancer cells by FACS sorting using E-cadherin. FIG. 3A shows the identification of surface markers for isolating tumor-initiating cells (cancer stem cells, CSC) from metastatic prostate cell lines. DU145, LNCaP and PC3 cells were immuno-stained for OCT3/4 (red), E-cadherin (green), DAPI (blue) and the merge of all pitures. Representative results of three independent experiments are shown. FIG. 3B depicts the sorting and analysis of prostate cancer stem cells and non-stem prostate cancer cells from the DU145 cell line by flowcytometry. Shown are the E-cadherin expression before (left) and after sorting of high expression (right, top) and low expression (right, bottom) tumor cells; the value in each graph represents the percentage of enriched population; dead cells were gated by propidium iodide (PI). Representative results of three experiments are shown. FIG. 3C depicts fluorescent-activated cell sorting (FACS) of stem cells sorted from DU145, LNCaP and PC3 prostate cell lines using E-cadherin as a marker. FIG. 3D shows RT-PCR analysis for the detection of mRNA levels of E-cadherin, Nanog and OCT3/4 in DU145, LNCaP and PC3 prostate cancer cell lines. Data were normalized to β-actin expression. Representative results of three independent experiments are shown.

FIGS. 4A-4F depict the isolation of stem cell-like prostate tumor cells by FACS sorting using E-cadherin. FIG. 4A shows the screening and identification of surface markers for isolating stem cell-like cells from prostate cell lines. DU145 and PC3 cells were immunostained for OCT3/4 (red), E-cadherin (green), DAPI (blue); OCT3/4 and E-cadherin (E-cad) staining were also merged. Magnification is 40× and the scale bar is 20 μm. FIGS. 4B and 4C are graphs depicting the phenotypic analysis of DU145 and PC3 cells using double-staining with E-cadherin and CD44 (FIG. 4B) or Integrin-α2β1 (FIG. 4C). Cells were gated on the E-cadherin+ (green) or E-cadherin− (blue) population. FIG. 4D is a graph depicting flow cytometry analysis of DU145 and PC3 cells showing E-cadherin expression. FIG. 4E is a graph depicting isotype matched controls of flow cytometry analysis of DU145 and PC3 used to set analysis gates for E-cadherin cell sorting. FIG. 4F shows RT-PCR analysis detecting expression levels of OCT3/4, SOX2, Nanog, c-Myc and Klf4 in E-cadherin⁺ and E-cadherin⁻ cells isolated from DU145 and PC3 cells. Data were normalized to β-actin expression. Representative results of three independent experiments are shown.

FIGS. 5A-5D show that prostate cancer stem cells isolated from metastatic prostate cancer cell lines are clonigenic, proliferative, can differentiate, and are invasive. FIG. 5A shows the clonigenic properites of prostate tumor stem cells (CSC) and non-stem prostate cancer cells (Non-CSC) in a colony forming assay. E-cadherin⁺ and E-cadherin⁻ cells isolated from metastatic prostate cancer cell lines by FACS analysis were cultured in semisolid medium of soft agar for 2-3 weeks until colonies were well-formed. The colonies were counted to determine the number of clones. Data represent the mean±SD from two independent experiments. **p<0.01. Representative plates from each group are shown in the insets above. FIG. 5B depicts representative images of spheroid culture assay using E-cadherin sorted cells. Western blot comparing unsorted parental line (P) to E-cadherin⁺ spheroids (S) showing protein levels of OCT3/4, SOX2, and E-cadherin. Data were normalized to β-actin expression. Magnification is 5×. FIG. 5C shows representative images of sorted prostate tumor stem cells (CSC) and non-stem prostate cancer cells (Non-CSC) on plates after 3 days culture. Representative phase images are on the left panels; representative immunofluorescence images detecting E-Cadherin are on the center panels and β-catenin on the right panels. Magnification is 5× for phase contrast and 40× for immunofluorescence. The scale bar is 100 μm for phase contrast and 20 μm for immunofluorescence. FIG. 5D shows representative images of a transwell migration assay demonstrating the invasiveness of prostate tumor stem cells (CSC) and non-stem prostate cancer cells (Non-CSC). Representative phase images at 10× magnification are on the top panels; representative phase images at 20× magnification are on the bottom panels.

FIGS. 6A-6C show that prostate cancer stem cells isolated from metastatic stem cell lines are tumorigenic in SCID mice. FIG. 6A shows photographs of xenograft tumors in mice (five mice per group) injected with prostate tumor stem cells (CSC) and non-stem prostate cancer cells (Non-CSC). FIG. 6B is a graph depicting the tumorigenic potential of isolated tumor-initiating cells from the PC3 prostate cancer cell line in SCID mice after subcutaneous injection (sorted E-cadherin⁺, blue diamond; E-cadherin⁻, pink square). Data concerning tumor volume are mean±SD from five mice in each group. Representative results of two experiments are shown. FIG. 6C is a graph depicting the tumorigenic potential of isolated tumor-initiating cells from the DU145 prostate cancer cell line in SCID mice after subcutaneous injection. Data concerning tumor volume are mean±SD from five mice in each group. Representative results of two experiments are shown.

FIGS. 7A and 7B show the expression of pluripotent stem cell genes in metastatic prostate tumor-initiating stem cells. FIG. 7A shows RT-PCR analysis for the detection of mRNA levels of c-Myc, Klf4, OCT3/4 and Sox2 in tumor-initiating cells (CSC) or non-tumor-initiating cells (NC) isolated from DU145, LNCaP and PC3 cells. Data were normalized to β-actin expression. Representative results of three independent experiments are shown. FIG. 7B shows Western-blot analysis for detecting protein levels of c-Myc, Klf4, Nanog, OCT3/4 and Sox2 in tumor-initiating cells isolated from DU145, LNCaP and PC3 cells. Data were normalized to β-actin expression. Representative results of three independent experiments are shown.

FIGS. 8A-8D show that prostate cancer stem cells are present in human prostate tumor tissue. FIG. 8A depicts a hypothetical model for the origin and differentiation of cancer stem cells in prostate. FIG. 8B shows RT-PCR analysis for the detection of mRNA levels of OCT3/4, Sox2, c-Myc, Nanog, prostate specific antigen (PSA) and androgen receptor (AR) in 4 independent samples of tumor tissue from primary human prostate cancer (PCa#1, PCa#2, PCa#3, PCa#4), isolated prostate cancer stem cells (Pca SC), embryonic stem cells (ESC) or dendritic cells (DC). Data were normalized to β-actin expression. Representative results of three independent experiments are shown. FIG. 8C shows the expression of OCT 3/4 (top panels) and SOX2 (lower panels) in human tissue samples visualized by immunohistochemical staining using antibodies specific for OCT 3/4 and SOX2 respectively. Representative images of normal prostate (left panels) and fetal testes (right panels) are shown. Images were captured with Zeiss Axioplan 2 upright microscope. Brown color stained cells represent the positive cells. FIG. 8D shows the expression of OCT 3/4 and SOX2 in human prostate tumor tissue. Representative images of prostate tumor tissue visualized by Hematoxylin and Eosin (H&E) staining (upper left panel), immunohistochemical staining with IgG contro antibodies (lower left panel), immunohistochemical staining with OCT 3/4 antibodies (upper right panel, magnification in inset), and immunohistochemical staining with SOX2 antibodies (lower right panel, magnification in inset). Images were captured with Zeiss Axioplan 2 upright microscope. Brown color stained cells represent the positive cells.

FIGS. 9A-9F depict expression of pluripotent stem cell genes c-Myc, Klf4, Nanog, OCT3/4 and Sox2 in prostate cancer tissues. FIGS. 9A-9E are graphs showing semiquantitative RT-PCR of OCT3/4 (FIG. 9A), Sox2 (FIG. 9B), Nanog (FIG. 9C), c-Myc (FIG. 9D), and Klf4 (FIG. 9E) using commerically available prostate tissue panels (Origene TissueScan). Normal prostate (N, black), prostate tumor sphere cells (PS, crosshatch) and hESC (ES, gray) served as controls. Band intensities were calculated using AlphaEase software (AlphaInnotech). Transcript levels for each case were normalized to β-actin expression and are represented as relative units standardized to the normal tissue pool. Representative results of three independent experiments are shown. Statistical significance was set at p<0.05; * is statistically different from the normal tissue pool and ‡ is statistically different from hESC. FIG. 9F is a graph depicting the correlation of mRNA expression levels between SOX2 and OCT3/4 in tissue samples. The relative level of SOX2 expression was plotted against the relative level of OCT3/4 expression using the normal tissue pool as reference and gave a Spearman correlation coefficient of 0.4730 (p<0.0001).

FIGS. 10A-10C depict the immunohistochemical detection of OCT3/4 and SOX2 in human prostate cancer tissues. FIG. 10A provides images of immunostaining for OCT3/4 and SOX2 using prostate tissue arrays. Representative images from negative, low (<5%), intermediate (5-25%) and high (26-50%) percentage staining are shown. Brown color indicates positive nuclear staining. Magnification is 20× with the inset at 40× and the scale bar is 70 μm. Representative results of at least two independent experiments are shown. FIGS. 10B and 10C are graphs classifying different Gleason Score samples based on category of staining intensity for OCT3/4 (FIG. 10B) and SOX2 (FIG. 10C). The red line represents the mean.

FIGS. 11A and 11B show that prostate cancer stem cells are resistant to irradiation. FIG. 11A shows Western blot analysis performed on the samples of prostate cancer stem cells using antibodies to Sox2, Oct3/4, Nanog, E-cadherin, β-Catenin and Actin in which the prostate cancer cells were exposed to various doses of radiation, including 0 Gy, 2 Gy, 4 Gy, 6 Gy, and 8 Gy doses. FIG. 11B is a graph depicting the surviving fraction of prostate cancer stem cells (CSC) and non-stem prostate cancer cells (Non-CSC) from the samples exposed to radiation (0 Gy, 2 Gy, 4 Gy, 6 Gy, 8 Gy and 10 Gy).

FIGS. 12A and 12B show that prostate cancer stem cells are resistant to Docetaxel. FIG. 12A shows Western blot analysis performed on samples of prostate cancer stem cells using antibodies to Sox2, Oct3/4, Nanog, E-cadherin, β-Catenin and Actin in which the prostate cancer cells were exposed to various doses of docetaxel, including 1 nM, 2 nM, 5 nM, and 10 nM doses. FIG. 12B is a graph depicting the cell viability of prostate cancer stem cells (CSC) and non-stem prostate cancer cells (Non-CSC) observed for up to 72 hours after treatment with Docetaxel. Cell viability was determined by quantifying the surviving prostate cancer stem cells (CSC) and non-stem prostate cancer cells (Non-CSC) in the samples exposed to 5 nM Docetaxel at Day 0, 1, 2 and 3.

FIGS. 13A-13C show that prostate cancer stem cells are immune privileged or immunosuppressive. FIG. 13A shows RT-PCR analysis for the detection of mRNA levels of LMP2, LMP7, TAP1, TAP2, and Tapasin in tumor-initiating stem cells (Ecad+) or non-tumor-initiating cells (Ecad−) isolated from DU145, LNCaP and PC3 cells. Data were normalized to β-actin expression. Representative results of three independent experiments are shown. FIG. 13B shows RT-PCR analysis for the detection of mRNA levels of CD44, Ecad, Nanog, OCT3/4, and TERT in tumor-initiating stem cells (CSC) or non-tumor-initiating cells (Non-CSC) isolated from LNCaP cells, which were used in the experiment in FIG. 13C. Data were normalized to β-actin expression. Representative results of three independent experiments are shown. FIG. 13C is a graph depicting the data from Interferon-γ enzyme linked immunosorbent spot (IFN-γ ELISPOT) assays performed on prostate cancer stem cells (CSC) and dendritic cells (DC). Prostate cancer stem cells were untreated (CSC), treated with isotype-specific antibody (CSC+Iso Ab.), treated with antibody to E-cadherin (CSC+E-cad blocking), or treated with antibody to HLA-class I (CSC+HLA Blocking). Untreated dendritic cells were used as a negative control and dendritic cells expressing hTERT (hTERT DC) were used as a positive control. Antigen-specific T-cells were mixed with the cells in the samples, plated, and the numbers spot forming colonies quantified for each sample.

FIGS. 14A-14B show that siRNAs to transciption factors in prostate stem cells increase cell death in prostate stem cells. FIG. 14A shows RT-PCR analysis examining the efficiency of siRNAs targeted against c-Myc, Klf4, Nanog, OCT3/4 and Sox2 in silencing mRNA expression of corresponding genes in prostate cancer stem cells. The mRNA levels of these genes in cells treated with control siRNA (Cntl-siR) were used as controls. Data were normalized to β-actin expression. Representative results of two independent experiments are shown. FIG. 14B shows flow cytometry analysis of CSC cells and Non-CSC cells from DU145, LNCaP and PC3 cells treated with siRNAs against c-Myc, Klf4, Nanog, OCT3/4 and Sox2 for 24 hours. Cells were recovered and apoptotic cells were detected using the annexin V and PI binding assay. Value in lower left corner represents the percentage of viable cells. Cells treated with control siRNA were used as controls. Representative results of three independent experiments are shown.

FIGS. 15A-15D show that shRNAs or siRNAs that reduce the expression of certain transciption factors in prostate stem cells also decrease the tumorigenicity of the prostate stem cells. FIG. 15A provides a Western blot showing decreased OCT3/4 or SOX2 protein levels in human DU145 prostate cancer cells transfected with shRNA compared to those transfected with control shRNA. FIG. 15B is a graph depicting the tumorigenic potential of isolated tumor-initiating stem cells from the DU145 cell line is decreased when DU145 prostate cancer stem cells are pre-treated with Sox2 or Oct 3/4 shRNA in SCID mice. Tumors were not detected in mice injected with stem cells pre-treated with Sox2 or Oct 3/4 shRNA just under 70 days after injection. Unsorted DU145 cells (1×10⁵) were subcutaneously injected in SCID mice after treatment with OCT 3/4 (pink square), SOX2 (green diamond), or control shRNA (blue triangle). Tumor volume data are reported as the mean±SD from the four mice that developed tumors. Representative results of two independent experiments are shown. FIG. 15C depicts representative images showing tumor development. The scale bar is 1 cm. FIG. 15D is a graph depicting the tumorigenic potential of isolated tumor-initiating stem cells from the DU145 cell line is decreased when DU145 prostate cancer stem cells are pre-treated with Sox2 or Oct 3/4 siRNA in SCID mice. Data concerning tumor volume are mean±SD from five mice in each group.

FIGS. 16A-16F show the identification of stem cells from human renal cancer cell lines and renal primary tumor tissue samples. FIG. 16A shows Western blot analysis for the detection of protein levels of OCT3/4, SOX2, Nanog, c-Myc, and Klf4 in renal cell carcinoma (RCC) cell lines 769P, A498, ACHN, Caki-1, and Caki-2. Data were normalized to GADPH expression. Representative results are shown. FIG. 16B shows 769P, A498, ACHN, Caki-1, and Caki-2 renal cancer cells immunostained for OCT3/4 (red), Nanog (green) and the merge of OCT3/4 and Nanog (Merge, yellow). Phase contrast images served as controls. Representative results of three independent experiments are shown. FIG. 16C shows 769P, A498, ACHN, Caki-1, and Caki-2 renal cancer cells immunostained for OCT3/4 (red), E-cadherin (Ecad green) and the merge of OCT3/4 and Nanog (Merge, yellow). Phase contrast images served as controls. Representative results of three independent experiments are shown. FIG. 16D depicts flow cytometry analysis of renal cancer stem cells and non-stem renal cancer cells from the 769P, ACHN, and Caki-1 cell lines using isotype antibodies (Isotype, left panels) or E-cadherin antibodies (Ecad, right panels) to label cells. The value in each graph represents the percentage of cells in the enriched population; dead cells were gated by propidium iodide (PI). Representative results of three experiments are shown. FIG. 16E shows RT-PCR analysis for the detection of mRNA levels of GAPDH, OCT3/4, Sox2, Nanog, Klf4, and c-Myc, in 4 independent samples of tumor tissue from renal cancer patients (JPGRCC, J; EJWRCCO12204-01, E; JWVRCC112603-001, W; and RNNRCCO81403-001, R), or human embryonic stem cells (hESC d21). FIG. 16F shows RT-PCR analysis for the detection of mRNA levels of GAPDH, OCT3/4, Sox2, Nanog, Klf4, and c-Myc, in 4 independent samples of tumor tissue from renal cancer patients or human embryonic stem cells (hESC).

FIGS. 17A-17C show the identification of stem cells from human bladder cancer cell lines. FIG. 17A shows 5637, HT1376, J82, and TCCSUP bladder cancer cells immunostained for OCT3/4 (red), Nanog (green) and the merge of OCT3/4 and Nanog (Merge, yellow). Phase contrast images served as controls. Representative results of three independent experiments are shown. FIG. 17B shows 5637, HT1376, J82, and TCCSUP bladder cancer cells immunostained for OCT3/4 (red), E-cadherin (Ecad green) and the merge of OCT3/4 and Nanog (Merge, yellow). Phase contrast images served as controls. Representative results of three independent experiments are shown. FIG. 17C depicts flowcytometry analysis of bladder cancer stem cells and non-stem bladder cancer cells from the HT1376, J82, and TCCSUP cell lines using isotype antibodies (Isotype, left panels) or E-cadherin antibodies (Ecad, right panels) to label cells. The value in each graph represents the percentage of cells in the enriched population; dead cells were gated by propidium iodide (PI). Representative results of three experiments are shown.

FIGS. 18A-18B show the identification of stem cells from various human and mouse cancer cell lines. FIG. 18A shows B16F0 and B16F10 mouse melanoma cells, KHT mouse sarcoma cells, and 4A4 and 2C5 human breast tumor cells immunostained for OCT3/4 (red), Nanog (green) and the merge of OCT3/4 and Nanog (Merge, yellow). Phase contrast images served as controls. Representative results of three independent experiments are shown. FIG. 18B shows B16F0 and B16F10 mouse melanoma cells, KHT mouse sarcoma cells, and 4A4 and 2C5 human breast tumor cells immunostained for OCT3/4 (red), E-cadherin (Ecad green) and the merge of OCT3/4 and Nanog (Merge, yellow). Phase contrast images served as controls. Representative results of three independent experiments are shown.

FIG. 19 provides exemplary sequences of human OCT3/4, Nanog, Sox2, c-Myc and Klf4 polypeptides and nucleic acid molecules.

### DETAILED DESCRIPTION OF THE INVENTION

The invention features compositions and methods that are useful for the diagnosis, treatment and prevention of neoplasias (e.g., prostate cancer, melanoma, renal carcinoma, bladder cancer, breast cancer) as well as for treatment selection. The present invention is based, at least in part, on the discovery that five pluripotent stem cell transcription factors, OCT3/4, Nanog, Sox2, c-Myc and Klf4, are expressed by tumor-initiating cells. Stem-like tumor-initiating cells were identified and isolated from primary prostate tumor tissue and three metastatic prostate tumor lines, and these cells exhibited a clear stem cell transcriptional signature. This discrete population of stem-like tumor-initiating cells possessed strong tumorgenicity and transplantability in SCID mice and are resistant to the radiation therapy and chemo-therapy. Furthermore, inhibition of any one of these genes in these cells resulted in significant apoptosis and necrosis. As reported in more detail below, prostate tumor-initiating cells may achieve pluripotency by reprogramming and expressing the combination of OCT3/4, Nanog, Sox2, c-Myc and Klf4 stem cell transcription factors.

### Prostate Cancer Stem Cells

The development of human prostate cancer proceeds through a series of defined stages, beginning with prostatic intraepithelial neoplasia, progressing to invasive hormone-dependent cancer, and finally progressing to hormone-independent cancer. Most human prostate cancers are adenocarcinomas that express markers associated with luminal epithelial cells. Because of unbalanced cell proliferation, cell differentiation, and cell death, prostate cancer exhibits substantial histological heterogeneity. To date, DNA and tissue microarrays of tumors have failed to account for cellular heterogeneity and differences in the proliferative potential of different populations within tumors. At present, all of the phenotypically diverse cancer cells are treated as though they have unlimited proliferative potential and can acquire the ability to metastasize. In patients with metastic disease, conventional therapies are ineffective. Metastatic prostate tumor cells are able to survive extreme conditions within the circulation. Metastic cancer cells lodge in the capillary beds of distant organs where they undergo extensive proliferation, often in bone, lymph node, lung and brain. Metastatic tumor cells share many characteristics (e.g., self-renewal, proliferation, and multi-potency) with pluripotent stem cells. Little is known about how human metastatic tumor cells maintain or acquire their multipotency. Recent studies suggest the existence of prostate cancer stem cells that are chemo-resistant and radiation-resistant. Therapies specifically directed against such cancer stem cells are likely to be more effective in curing prostate cancer and metastatic disease.

Accordingly, the present invention provides methods of treating prostate cancer and/or disorders or symptoms thereof which comprise administering a therapeutically effective amount of a pharmaceutical composition comprising an agent of the formulae herein to a subject (e.g., a mammal, such as a human). Thus, one embodiment is a method of treating a subject suffering from or susceptible to prostate cancer, metastatic prostate cancer, or prostate cancer having the propensity to metastasize or symptoms thereof. The method includes the step of administering to the mammal a therapeutic amount of an agent herein sufficient to treat the prostate cancer or symptom thereof, under conditions such that the prostate cancer is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the agents herein, such as an agent of the formulae herein to a subject (e.g., animal, human) in need thereof, including a mammal, particularly a human. Such treatment will be suitably administered to subjects, particularly humans, suffering from, having, susceptible to, or at risk for prostate cancer, including metastatic disease or prostate cancer having a propensity to metastasize, or a symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which prostate cancer or hyperplasia may be implicated.

In one embodiment, the invention provides a method of monitoring treatment progress. The method includes the step of determining a level of diagnostic marker (Marker) (e.g., OCT3/4, Nanog, Sox2, c-Myc, Klf4 or any target delineated herein modulated by a compound herein, a protein or indicator thereof, etc.) or diagnostic measurement (e.g., screen, assay) in a subject suffering from or susceptible to prostate cancer, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Therapeutic Uses

The present invention features methods for treating cancer or the progression of cancer by administering OCT3/4, NANOG, SOX2, C-MYC or KLF4 inhibitory nucleic acid molecules or agents that decrease the expression or biological activity of an OCT3/4, NANOG, SOX2, C-MYC or KLF4 nucleic acid molecule or polypeptide. Advantageously, such agents selectively target prostate tumor initiating stem cells. Compounds of the present invention may be administered by any appropriate route for the treatment or prevention of neoplasia. These may be administered to humans, domestic pets, livestock, or other animals with a pharmaceutically acceptable diluent, carrier, or excipient, in unit dosage form. Administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in Remington: The Science and Practice of Pharmacy (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

### Inhibitory Nucleic Acids

Inhibitory nucleic acid molecules are those oligonucleotides that inhibit the expression or activity of a OCT3/4, Nanog, Sox2, c-Myc or Klf4 polypeptide. Such oligonucleotides include single and double stranded nucleic acid molecules (e.g., DNA, RNA, and analogs thereof) that bind a nucleic acid molecule that encodes a OCT3/4, Nanog, Sox2, c-Myc or Klf4 polypeptide (e.g., antisense molecules, siRNA, shRNA) as well as nucleic acid molecules that bind directly to a OCT3/4, Nanog, Sox2, c-Myc or Klf4 polypeptide to modulate its biological activity (e.g., aptamers).

Ribozymes

Catalytic RNA molecules or ribozymes that include an antisense OCT3/4, Nanog, Sox2, c-Myc or Klf4 sequence of the present invention can be used to inhibit expression of a OCT3/4, Nanog, Sox2, c-Myc or Klf4 nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of an OCT3/4, Nanog, Sox2, c-Myc or Klf4 gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of OCT3/4, Nanog, Sox2, c-Myc or Klf4 expression. In one embodiment, OCT3/4, Nanog, Sox2, c-Myc or Klf4 expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Assays for Measuring Cell Viability

Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that induce or increase neoplastic cell death (e.g., increase apoptosis, reduce cell survival) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Neoplastic cells have a propensity to metastasize, or spread, from their locus of origination to distant points throughout the body. Assays for metastatic potential or invasiveness are known to the skilled artisan. Such assays include in vitro assays for loss of contact inhibition (Kim et al., Proc Natl Acad Sci USA. 101:16251-6, 2004), increased soft agar colony formation in vitro (Zhong et al., Int J. Oncol. 24(6):1573-9, 2004), pulmonary metastasis models (Datta et al., In Vivo, 16:451-7, 2002) and Matrigel-based cell invasion assays (Hagemann et al. Carcinogenesis. 25: 1543-1549, 2004). In vivo screening methods for cell invasiveness are also known in the art, and include, for example, tumorigenicity screening in athymic nude mice. A commonly used in vitro assay to evaluate metastasis is the Matrigel-Based Cell Invasion Assay (BD Bioscience, Franklin Lakes, N.J.).

If desired, candidate compounds selected using any of the screening methods described herein are tested for their efficacy using animal models of neoplasia. In one embodiment, mice are injected with neoplastic human cells. The mice containing the neoplastic cells are then injected (e.g., intraperitoneally) with vehicle (PBS) or candidate compound daily for a period of time to be empirically determined. Mice are then euthanized and the neoplastic tissues are collected and analyzed for OCT3/4, Nanog, SOX2, c-Myc or Klf4 mRNA or protein levels using methods described herein. Compounds that decrease Oct3/4, Nanog, Sox2, c-Myc or Klf4 mRNA or protein expression relative to control levels are expected to be efficacious for the treatment of a neoplasm in a subject (e.g., a human patient). In another embodiment, the effect of a candidate compound on tumor load is analyzed in mice injected with a human neoplastic cell. The neoplastic cell is allowed to grow to form a mass. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. As used herein, the term “prostate cancer” is meant a collection of prostate cells multiplying in an abnormal manner. Cancer growth is uncontrolled and progressive, and occurs under conditions that would not elicit, or would cause cessation of, multiplication of normal cells.

A nucleobase oligomer of the invention, or other negative regulator of OCT3/4, Nanog, Sox2, c-Myc or Klf4, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for delivering an agent that disrupts the activity of OCT3/4, Nanog, Sox2, c-Myc and Klf4 polypeptides or polynucleotides include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleobase oligomer of the invention is likely to depend on such variables as the type and extent of the disorder, the overall health status of the particular patient, the formulation of the compound excipients, and its route of administration.

As described above, if desired, treatment with a nucleobase oligomer of the invention may be combined with therapies for the treatment of proliferative disease (e.g., radiotherapy, surgery, or chemotherapy).

For any of the methods of application described above, a nucleobase oligomer of the invention is desirably administered intravenously or is applied to the site of the needed apoptosis event (e.g., by injection).

### Oligonucleotides and Other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with an OCT3/4, Nanog, Sox2, c-Myc or Klf4 nucleic acid molecule. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FESS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Polynucleotide Therapy

Polynucleotide therapy is another therapeutic approach in which a nucleic acid encoding a OCT3/4, NANOG, SOX2, C-MYC or KLF4 inhibitory nucleic acid molecule is introduced into cells. The transgene is delivered to cells in a form in which it can be taken up and expressed in an effective amount to inhibit neoplasia progression.

Transducing retroviral, adenoviral, or human immunodeficiency viral (HIV) vectors are used for somatic cell gene therapy because of their high efficiency of infection and stable integration and expression (see, for example, Cayouette et al., Hum. Gene Ther., 8:423-430, 1997; Kido et al., Curr. Eye Res. 15:833-844, 1996; Bloomer et al., J. Virol. 71:6641-6649, 1997; Naldini et al., Science 272:263-267, 1996; Miyoshi et al., Proc. Natl. Acad. Sci. USA, 94:10319-10323, 1997). For example, OCT3/4, NANOG, SOX2, C-MYC or KLF4 inhibitory nucleic acid molecules, or portions thereof, can be cloned into a retroviral vector and driven from its endogenous promoter, from the retroviral long terminal repeat, or from a promoter specific for the target cell type of interest (such as epithelial carcinoma cells). Other viral vectors that can be used include, but are not limited to, adenovirus, adeno-associated virus, vaccinia virus, bovine papilloma virus, vesicular stomatitus virus, or a herpes virus such as Epstein-Barr Virus.

Gene transfer can be achieved using non-viral means requiring infection in vitro. This would include calcium phosphate, DEAE-dextran, electroporation, and protoplast fusion. Liposomes may also be potentially beneficial for delivery of DNA into a cell. Although these methods are available, many of these are of lower efficiency.

### Screening Methods

The invention provides methods for identifying agents useful for the treatment or prevention of prostate cancer. Screens for the identification of such agents employ prostate cancer stem cells identified according to the methods of the invention. The use of such cells, which express increased levels of OCT3/4, NANOG, SOX2, C-MYC and/or KLF4 is particularly advantageous for the identification of agents that reduce the survival of this aggressive subpopulation of prostate cancer cells. Agents identified as reducing the survival, reducing the proliferation, or increasing cell death in OCT3/4, NANOG, SOX2, C-MYC or KLF4 expressing cell are particularly useful.

Methods of observing changes in OCT3/4, NANOG, SOX2, C-MYC or KLF4 interactions and OCT3/4, NANOG, SOX2, C-MYC or KLF4 biological activity are exploited in high throughput assays for the purpose of identifying compounds that modulate OCT3/4, NANOG, SOX2, C-MYC or KLF4 biological activity, e.g., transciptional regulation or protein-nucleic acid interactions. Compounds that inhibit OCT3/4, NANOG, SOX2, C-MYC or KLF4 binding to a regulated gene, or that inhibit another OCT3/4, NANOG, SOX2, C-MYC or KLF4 biological activity (e.g., OCT3/4, NANOG, SOX2, C-MYC or KLF4 's activity as a transcriptional activator or repressor), may be identified by such assays. In addition, compounds that modulate the expression of a OCT3/4, NANOG, SOX2, C-MYC or KLF4 polypeptide or nucleic acid molecule whose expression is altered in a patient having a neoplasia may be identified.

Any number of methods are available for carrying out screening assays to identify new candidate compounds that decrease the expression of an OCT3/4, NANOG, SOX2, C-MYC or KLF4 nucleic acid molecule. In one example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound which reduces the expression of a OCT3/4, NANOG, SOX2, C-MYC or KLF4 gene, or a functional equivalent thereof, is considered useful in the invention; such a molecule may be used, for example, as a therapeutic to treat a neoplasia in a human patient.

In another example, the effect of candidate compounds may be measured at the level of polypeptide production using the same general approach and standard immunological techniques, such as Western blotting or immunoprecipitation with an antibody specific for a polypeptide encoded by an OCT3/4, NANOG, SOX2, C-MYC or KLF4 gene. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a neoplasia in a human patient.

In yet another working example, candidate compounds may be screened for those that specifically bind to a polypeptide encoded by an OCT3/4, NANOG, SOX2, C-MYC or KLF4 gene. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention. In another embodiment, a candidate compound is tested for its ability to inhibit the biological activity of a polypeptide described herein, such as a OCT3/4, NANOG, SOX2, C-MYC or KLF4 polypeptide. The biological activity of an OCT3/4, NANOG, SOX2, C-MYC or KLF4 polypeptide may be assayed using any standard method, for example, a matrigel cell invasion or cell migration assay.

In another working example, a nucleic acid described herein (e.g., an OCT3/4, NANOG, SOX2, C-MYC or KLF4 nucleic acid) is expressed as a transcriptional or translational fusion with a detectable reporter, and expressed in an isolated cell (e.g., mammalian) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of a neoplasia. Preferably, the compound decreases the expression of the reporter.

In another example, a candidate compound that binds to a polypeptide encoded by an OCT3/4, NANOG, SOX2, C-MYC or KLF4 gene may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the OCT3/4, NANOG, SOX2, C-MYC or KLF4 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the activity of an OCT3/4, NANOG, SOX2, C-MYC or KLF4 polypeptide (e.g., as described herein). Compounds isolated by this approach may also be used, for example, as therapeutics to treat a neoplasia in a human patient. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to a nucleic acid sequence or polypeptide of the invention (e.g., an OCT3/4, NANOG, SOX2, C-MYC or KLF4 polypeptide or nucleic acid molecule).

Each of the DNA sequences listed herein may also be used in the discovery and development of a therapeutic compound for the treatment of neoplasia. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Optionally, compounds identified in any of the above-described assays may be confirmed as useful in an assay for compounds that modulate the propensity of a neoplasia to metastasize.

Small molecules of the invention preferably have a molecular weight below 2,000 daltons, more preferably between 300 and 1,000 daltons, and most preferably between 400 and 700 daltons. It is preferred that these small molecules are organic molecules.

### Test Extracts and Agents

In general, agents that modulate OCT3/4, NANOG, SOX2, C-MYC or KLF4 expression, biological activity, or OCT3/4, NANOG, SOX2, C-MYC or KLF4-dependent signaling are identified from large libraries of both natural products, synthetic (or semi-synthetic) extracts or chemical libraries, according to methods known in the art. Preferably, these compounds decrease OCT3/4, NANOG, SOX2, C-MYC or KLF4 expression or biological activity.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.), Aldrich Chemical (Milwaukee, Wis.), and Talon Cheminformatics (Acton, Ont.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

### Diagnostic Assays

The present invention provides a number of diagnostic assays that are useful for the identification or characterization of prostate cancer in a subject. Such methods may be used alone or in combination with standard methods for determining the stage or grade of prostate cancer. In one embodiment, a neoplasia is characterized by quantifying or determining the level of OCT3/4, Nanog, Sox2, c-Myc and Klf4 polypeptides or polynucleotide in the neoplasia. If desired, the levels of one, two, three, four or all of OCT3/4, Nanog, Sox2, c-Myc and Klf4 is measured. The expression of increased levels of all four of these Markers is indicative of a prostate cancer that has metastasized or that has the propensity to metastasize.

The level of any one or more of the OCT3/4, Nanog, Sox2, c-Myc and Klf4 is used, alone or in combination with other standard methods, to determine the stage or grade of a neoplasia. Grading is used to describe how abnormal or aggressive the neoplastic cells appear, while staging is used to describe the extent of the neoplasia. If desired, the grade and stage of the neoplasia in combination with the level of expression of OCT3/4, Nanog, Sox2, c-Myc and Klf4 is used to determine a subject's long-term prognosis (i.e., probable response to treatment and survival). Thus, the methods of the invention are useful for predicting a patient's prognosis, and for selecting a course of treatment.

The Gleason scale is the most common scale used for grading prostate cancer. A pathologist will look at the two most poorly differentiated parts of the tumor and grade them. The Gleason score is the sum of the two grades, and so can range from two to 10. The higher the score is, the poorer the prognosis. Scores usually range between 4 and 7. The scores can be broken down into three general categories: (i) low-grade neoplasias (score<4) are typically slow-growing and contain cells that are most similar to normal prostate cells; intermediate grade neoplasias (4<score<7) are the most common and typically contain some cells that are similar to normal prostate cells as well as some more abnormal cells; high-grade neoplasias (8<score<10) contain cells that are most dissimilar to normal prostate cells. High-grade neoplasias are the most deadly because they are most aggressive and fast growing. High-grade neoplasias typically move rapidly into surrounding tissues, such as lymph nodes and bones.

Stage refers to the extent of a cancer. In prostate cancer, for example, one staging method divides the cancer into four categories, A, B, C, and D. Stage A describes a cancer that is only found by elevated PSA and biopsy, or at surgery for obstruction. It is not palpable on digital rectal exam (DRE). This stage is localized to the prostate. This type of cancer is usually curable, especially if it has a relatively low Gleason grade. Stage B refers to a cancer that can be felt on rectal examination and is limited to the prostate. Bone scans or CT/MRI scans are often used to determine this stage, particularly if prostate specific antigen (PSA) levels are significantly elevated or if the Gleason grade is 7 or greater. Many Stage B prostate cancers are curable. Stage C cancers have spread beyond the capsule of the prostate into local organs or tissues, but have not yet metastasized to other sites. This stage is determined by DRE, or CT/MRI scans, and/or sonography. In Stage C a bone scan or a PROSTASCINT scan is negative. Some Stage C cancers are curable. Stage D cancer has metastasized to distant lymph nodes, bones or other sites. This is usually determined by bone scan, PROSTASCINT scan, or other studies. Stage D cancer is usually incurable, but are expected to be treatable using the methods of the invention, i.e., by disrupting the expression or activity of one or more of OCT3/4, Nanog, Sox2, c-Myc and Klf4.

### Types of Biological Samples

The level of OCT3/4, Nanog, Sox2, c-Myc and Klf4 polypeptide or polynucleotide expression can be measured in different types of biologic samples. In one embodiment, the biologic sample is a tissue sample that includes cells of a tissue or organ (e.g., prostatic tissue cells). Prostatic tissue is obtained, for example, from a biopsy of the prostate. In another embodiment, the biologic sample is a biologic fluid sample. Biological fluid samples include blood, blood serum, plasma, urine, seminal fluids, and ejaculate, or any other biological fluid useful in the methods of the invention.

### Microarrays

The invention provides diagnostic microarrays for measuring the expression of OCT3/4, Nanog, Sox2, c-Myc and Klf4 in a biological sample. OCT3/4, Nanog, Sox2, c-Myc and Klf4 nucleic acid molecules or polypeptides are useful as hybridizable array elements in the microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al. (Nature Genet. 26:283-289), and in U.S. Pat. No. 6,436,665, hereby incorporated by reference.

### Nucleic Acid Microarrays

To produce a nucleic acid microarray oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, cerebrospinal fluid, phlegm, saliva, or urine) or tissue sample (e.g. a tissue sample obtained by biopsy). For some applications, cultured cells (e.g., lymphocytes) or other tissue preparations may be used. The mRNA is isolated according to standard methods, and cDNA is produced and used as a template to make complementary RNA suitable for hybridization. Such methods are described herein. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 3° C., more preferably of at least about 37 C., and most preferably of at least about 42 C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30.degree. C. in 750 mM NaCl, 75 mM trisodium citrate, and 1% SDS. In a more preferred embodiment, hybridization will occur at 37.degree. C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42.degree. C. in 250 mM NaCl, 25 mM trisodium citrate, 1% SDS, 50% formamide, and 200 .mu.g/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 C., more preferably of at least about 42 C., and most preferably of at least about 68 C. In a preferred embodiment, wash steps will occur at 25 C. in 30 mM NaCl, 3 mM trisodium citrate, and 0.1% SDS. In a more preferred embodiment, wash steps will occur at 42 C. in 15 mM NaCl, 1.5 mM trisodium citrate, and 0.1% SDS. In a most preferred embodiment, wash steps will occur at 68 C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

### Protein Microarrays

Proteins, such as those described herein, may also be analyzed using protein microarrays. Such arrays are useful in high-throughput low-cost screens to identify peptide or candidate compounds that bind a polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides encoded by a nucleic acid molecule listed in Table 2 or Table 4 or antibodies against such polypeptides) are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., prostate cancer cells). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Selection of a Treatment Method

After a subject is diagnosed as having prostate cancer, a method of treatment is selected. In prostate cancer, for example, a number of standard treatment regimens are available. The level of OCT3/4, Nanog, Sox2, c-Myc and Klf4 polypeptide or polynucleotide expression is used in selecting a treatment method. In one embodiment, less aggressive neoplasias have lower levels of OCT3/4, Nanog, Sox2, c-Myc and Klf4 expression than more aggressive neoplasias. In another embodiment, the expression profile of a neoplasia, or the level of expression of OCT3/4, Nanog, Sox2, c-Myc and Klf4 is correlated with a clinical outcome using statistical methods to determine the aggressiveness of the neoplasia. In one embodiment, a cell that expresses increased levels of each of OCT3/4, Nanog, Sox2, c-Myc and Klf4 relative to a reference (i.e., a healthy cell, or a prostate cancer cell that lacks a propensity to metastasize) correlates with a poor clinical outcome, such as metastasis or death. In other embodiments, the level of increase in OCT3/4, Nanog, Sox2, c-Myc and Klf4 expression is indicative of a poor prognosis, i.e., a 30%-60% increase over levels of OCT3/4, Nanog, Sox2, c-Myc and Klf4 expression in a control cell identifies the prostate cancer as an aggressive prostate cancer. The profile of OCT3/4, Nanog, Sox2, c-Myc and Klf4 expression (i.e., the expression of fewer than all five) or a slight increase in the level of expression (e.g., a 1-5% or 5-10% increase in the expression of one or more of OCT3/4, Nanog, Sox2, c-Myc and Klf4) correlates with a good clinical outcome. Such prostate cancers are identified as less aggressive.

Less aggressive prostate cancers are likely to be susceptible to conservative treatment methods. Conservative treatment methods include, for example, cancer surveillance, which involves periodic patient monitoring using diagnostic assays of the invention, alone or in combination, with PSA blood tests and DREs, or hormonal therapy. Cancer surveillance is selected when diagnostic assays indicate that the adverse effects of treatment (e.g., impotence, urinary, and bowel disorders) are likely to outweigh therapeutic benefits.

More aggressive neoplasias are less susceptible to conservative treatment methods. When methods of the invention indicate that a neoplasia is very aggressive, an aggressive method of treatment should be selected. Aggressive therapeutic regimens typically include one or more of the following therapies: radical prostatectomy, radiation therapy (e.g., external beam and brachytherapy), hormone therapy, and chemotherapy.

Methods of the invention may be used alone or in combination with conservative or aggressive therapeutic regimens to treat a prostate cancer that expresses one or more of OCT3/4, Nanog, Sox2, c-Myc and Klf4.

### Patient Monitoring

The disease state or treatment of a patient having neoplasia can be monitored using the methods and compositions of the invention. In one embodiment, a microarray is used to assay the expression profile of an OCT3/4, NANOG, SOX2, C-MYC or KLF4 nucleic acid molecule. Such monitoring may be useful, for example, in assessing the efficacy of a particular drug in a patient. Therapeutics that decrease the expression of at least one OCT3/4, NANOG, SOX2, C-MYC or KLF4 nucleic acid molecule or polypeptide are taken as particularly useful in the invention.

While methods of neoplasia treatment vary depending on the type of neoplasia, the stage of neoplasia, and the patient's age, health, and physical condition, more aggressive treatment regimens will be used in patients having a poor prognosis (e.g., patients having a metastatic prostate carcinoma or a prostate carcinoma with metastatic potential). As described above, the methods of the invention are useful in determining the prognosis of a patient having neoplasia, such as a neoplasia with increased metastatic potential. In such patients aggressive therapies may be used. These include therapies having increased toxicity and those having an increased risk of adverse side-effects. Aggressive therapies are employed earlier and at higher doses in patients having a poor prognosis.

### Combination Therapies

OCT3/4, NANOG, SOX2, C-MYC or KLF4 inhibitory nucleic acids may be administered alone or in any combination that is effective to treat a neoplasia. If desired, agents of the invention are administered in combination with any other standard neoplasia therapy; such methods are known to the skilled artisan (e.g., Wadler et al., Cancer Res. 50:3473-86, 1990), and include, but are not limited to, chemotherapy, hormone therapy, immunotherapy (include, but are not limited to, immunotherapy that will specifically target cancer stem cell transcription factors), radiotherapy, and any other therapeutic method used for the treatment of neoplasia.

### Kits

The invention provides kits for the treatment or prevention of prostate cancer, particularly prostate cancer that expresses one, two, three, four, or all of OCT3/4, Nanog, Sox2, c-Myc and Klf4. In one embodiment, the kit includes a therapeutic or prophylactic composition containing an effective amount of an inhibitory nucleic acid molecule that disrupts the expression of an OCT3/4, Nanog, Sox2, c-Myc and/or Klf4 polynucleotide or polypeptide in unit dosage form. In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an inhibitory nucleic acid molecule of the invention is provided together with instructions for administering the inhibitory nucleic acid molecule to a subject having or at risk of developing prostate cancer. The instructions will generally include information about the use of the composition for the treatment or prevention of prostate cancer. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### OCT 3/4, Nanog, Sox2, c-Myc, and Klf4 are Expressed in Cancer Stem Cells in Metastatic Prostate Cancer Cell Lines

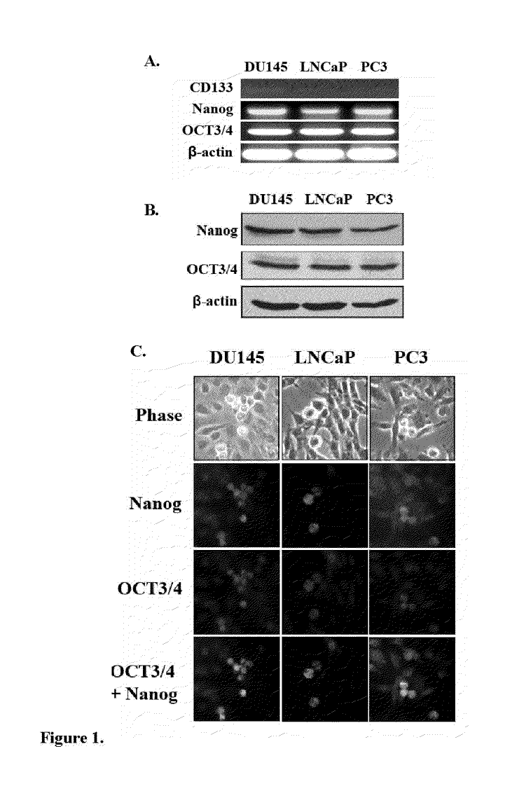

Self-renewal is a unique property shared by both normal and cancer stem cells. Cancer stem cells were examined for transcriptional characteristics of embryonic stem cells. Embryonic stem cell transcription factors OCT3/4 and Nanog, which are responsible for maintaining self-renewal and pluripotency of undifferentiated embryonic stem cells, were used as markers to identify cancer stem cells in metastatic prostate cancer cell lines. Reverse transcription Polymerase Chain Reaction (RT-PCR) was performed by standard methods to detect the mRNA levels of these genes in DU145, LNCaP and PC3 cells. OCT3/4 and Nanog expression were clearly detected at high levels in all of the prostate cancer cell lines (FIG. 1A). CD133 expression was also examined because variant solid tumor stem cells have been isolated using CD133 which is expressed on the cell surface (Collins et al., 2005; O'Brien et al., 2007; Singh et al., 2004). RT-PCR analysis showed that the expression of CD133 was very low or absent in DU145, LNCaP and PC3 tumor cell lines. Western blot analysis also confirmed the expression of OCT3/4 and Nanog proteins in the three prostate cancer cell lines (FIG. 1B).

To examine whether OCT3/4 and Nanog were expressed as proteins by putative cancer stem cells in the tumor cell lines, DU145, LNCaP and PC3 cultured cells were examined for OCT3/4 and Nanog expression by immunofluorescence staining. A distinct population of cells displayed high expression of both OCT3/4 and Nanog by immunofluorescence microscopy (FIG. 1C). These cells were readily observed in all three cell lines, representing 5-10% of total cells. These results suggest the existence of a small population of cancer stem cells in DU145, LNCaP and PC3 metastatic prostate tumor cell lines.

To investigate whether pluripotent stem cell reprogramming factors in addition to OCT3/4 and

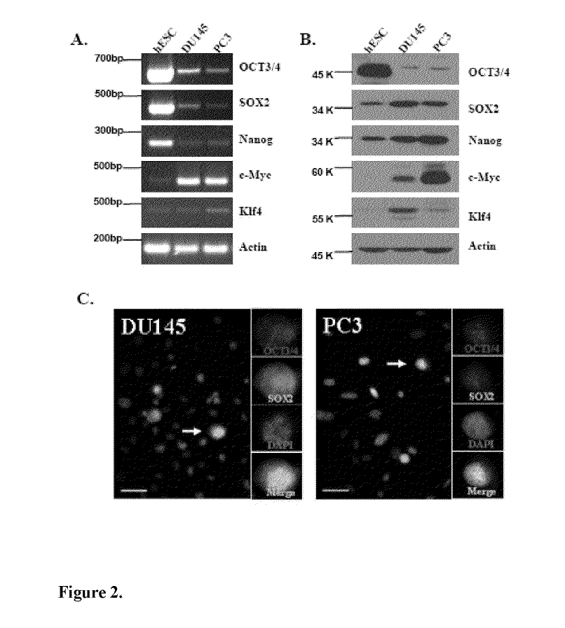

Nanog were expressed by a subpopulation of putative stem cell-like tumor cells in the prostate cancer cell lines, RT-PCR was used to evaluate the mRNA expression levels of the core pluripotent stem cell reprogramming factors SOX2, c-Myc, and Klf4 in DU145 and PC3 prostate cancer cell lines. In these studies, human embryonic stem cell (hESC) line H9 was used as a reference. RT-PCR analysis revealed that both prostate tumor cell lines expressed detectable levels of mRNA for SOX2, c-Myc, and Klf4 as well as OCT3/4, Nanog, (FIG. 2A). Additionally, OCT 3/4 transcripts were confirmed and shown not to be those of the related pseudogenes, as assessed by the method of Panagopoulos et. al. (2008. Genes Chromosomes Cancer 47:521-529). Compared to the embryonic stem cell line H9, the prostate tumor cell lines displayed relatively low levels of OCT3/4, SOX2 and Nanog. They did however express high levels of the oncogene c-Myc. Expression of Klf4, a context-dependent oncogene (15), was also elevated in prostate tumor cells compared to embryonic stem cells.

In addition to gene expression analysis, the five reprogramming factors were measured by Western blot analysis in the prostate tumor cell lines (FIG. 2B). Similar to the RT-PCR results for mRNA expression, DU145 and PC3 prostate tumor cells had lower expression of OCT3/4 protein and higher expression of c-Myc and Klf4 proteins than embryonic stem (ES) cells. Western blot analysis of SOX2 and Nanog revealed similar or higher levels of protein expression in the tumor cell lines compared to the normal ES cells when compared with RT-PCR analysis of mRNA expression. Taken together these data indicate that pluripotent stem cell reprogramming factors were activated in prostate cancer cells.

To investigate what population of putative stem cell-like tumor cells express OCT3/4 and SOX2 transcription factors in DU145 and PC3 prostate cancer cell lines, DU145 and PC3 cultured cells were examined for OCT3/4 and SOX2 expression by immunofluorescence staining. Immunofluorescent double staining for the two markers, showed that only a discrete population of tumor cells (−5-10% of total cells) stained positive for both OCT3/4 and SOX2 in the prostate tumor cell lines, (FIG. 2C) similar results were observed with immunofluorescence staining of Nanog and OCT3/4. These results indicated the existence of a small population of cancer stem cells in metastatic prostate tumor cell lines.

### Example 2

### Cancer Stem Cells Isolated from Metastatic Prostate Tumor Cell Lines Possessed High Tumorgenicity

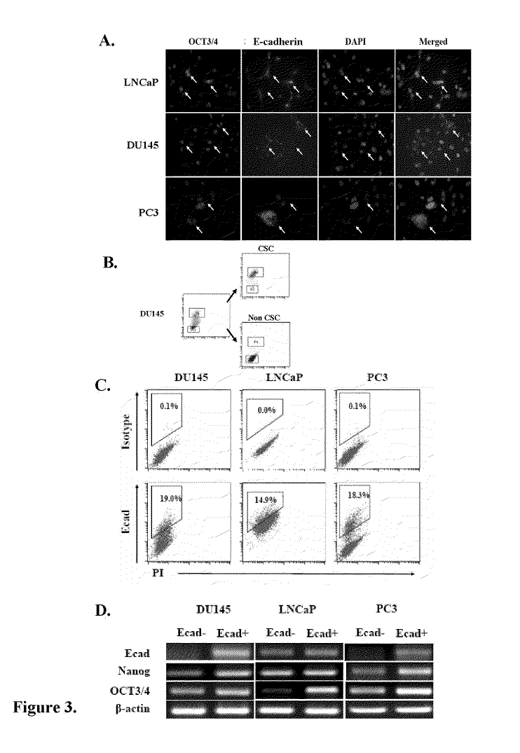

To isolate cancer stem cells from the prostate cancer cell lines, cell surface markers were screened by immunofluorescence microscopy for expression in the metastatic prostate cancer stem cells. DU145, LNCaP and PC3 cell lines were analyzed with an antibody panel of selected cell surface associated proteins in pluripotent stem cells and cancer stem cells, including CD9, E-cadherin, PODXL, SSEA1, SSEA4, CD24, and CD133. The tumor cell lines were also analyzed for the stem cell marker OCT3/4 to detect the cancer stem cells. Of the cell surface markers screened by co-immunofluorescence among the DU145, LNCaP and PC3 cell lines, E-cadherin showed high levels of expression in cells also expressing OCT3/4 (FIG. 3A). The expression levels of all the other markers tested did not correlate with OCT3/4 expression among the three prostate cancer cell lines. These results were further confirmed with flow cytometry-based analysis which showed that PC3 stem cells could be sorted from non-stem cancer cells (FIG. 3B). Based on these results, stem cell populations were separated from the three prostate cancer cell lines based on their E-cadherin expression profile using FACS analysis (Becton Dickinson MoFlo cell sorter) (FIG. 3C). In addition to the FACS analysis, the cell sorting method was further validated with RT-PCR assays using primers specific for E-cadherin, Nanog and OCT3/4. FACS sorting resulted in an enriched cancer cell population exhibiting high mRNA levels for the stem cell transcription factors Nanog and OCT3/4 as measured by RT-PCR (FIG. 3D).

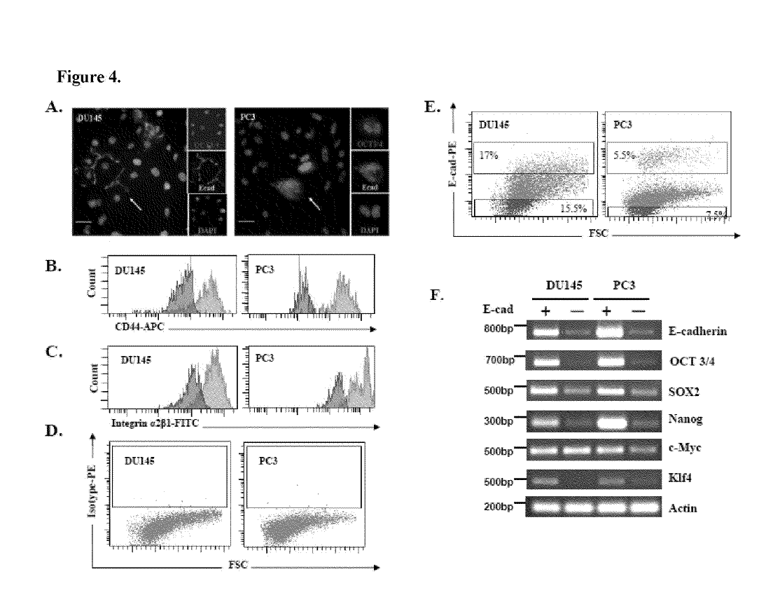

To assess the function of stem cell-like tumor cells that express pluripotent stem cell transcription factors in prostate cancer, potential cell surface markers of prostate stem cell-like tumor cells for cell sorting were screened in DU145 and PC3. The key stem cell regulator OCT3/4 was used as a marker to identify tumor cells with highly elevated stem-cell reprogramming factors. Once identified, these cells were then co-stained with a panel of cell surface antibodies selected on the basis of their association with pluripotent stem cells and cancer stem cells. These included CD44, ESA, and Integrin-α2β1 in addition to CD9, E-cadherin, PODXL, SSEA1, SSEA4 CD24, CD133, described above. The results showed that OCT3/4 nuclear positive cells were exclusively located in colonies that displayed classic morphology as malignant holoclones comprised of groups of tightly packed smaller tumor cells (Li et al., 2008. Cancer Res. 68:1820-1825.). Like holoclones from other carcinoma-derived cell lines, the holoclones from prostate tumor cell lines exhibited high expression of the epithelial marker E-cadherin (Locke et al., 2005. Cancer Res. 65:8944-8950). Indeed, most of OCT3/4 positive cells in the prostate tumor cell lines had high surface expression of E-cadherin (FIG. 4A). E-cadherin low or negative colonies contained few OCT3/4 positive tumors cells. Interestingly, PC3, which is known to have reduced surface E-cadherin expression due to the deletion of α-catenin gene, also displayed co-localized nuclear OCT3/4 staining with cytoplasmic E-cadherin staining (Morton et al., 1993. Cancer Res. 53:3585-3590). All other surface markers evaluated were detected at varying expression levels among the prostate cancer cell lines but these did not co-localize with OCT3/4 staining.

Importantly, E-cadherin positive cells exhibited not only OCT3/4 positive staining but also high expression of CD44 and Integrin-α2β1 as measured by flow cytometry analysis (FIGS. 4B and 4C). In both DU145 and PC3 cells expression of CD44 is exceedingly high (˜90% DU145, ˜100% PC3), making it difficult to isolate the stem cell population using only this marker. Investigators studying prostate cancer cell lines therefore typically have turned to using CD44 in combination with other markers including CD24, CD133, and Integrin-α2β1.

Putative stem cell-like populations were isolated from both prostate cancer cell lines by flow cytometry on the basis of the E-cadherin expression profiles. In these studies 17% of DU145 cells and 5.5% of PC3 cells were found to be positive for E-cadherin based on the isotype control (FIGS. 4D and 4E). Highly purified sub-populations of cells were obtained by isolating the top 5-10% of the cells highly expressing E-cadherin or the bottom 5-10% without E-cadherin expression. To confirm the enrichment of stem cell-like tumor cells after cell sorting, the gene expression of pluripotent stem cell reprogramming factors in the E-cadherin⁺ and E-cadherin⁻ populations was examined at the mRNA level (FIG. 4F). The data showed that compared to E-cadherin⁻ cells, only the E-cadherin⁺ cells expressed all five essential pluripotent stem cell reprogramming factors: OCT3/4, SOX2, Nanog, c-Myc and Klf4. Thus, E-cadherin, which showed distinguishable expression in the two cell lines (˜17% DU145 and ˜5.5% PC3), was utilized as a solitary, reliable and discrete marker for isolating the stem-like cell population from prostate cancer cell lines. These results indicated that E-cadherin can serve as a distinct surface marker to isolate prostate tumor initiating cells in these two cell lines and does not require combinatorial staining.

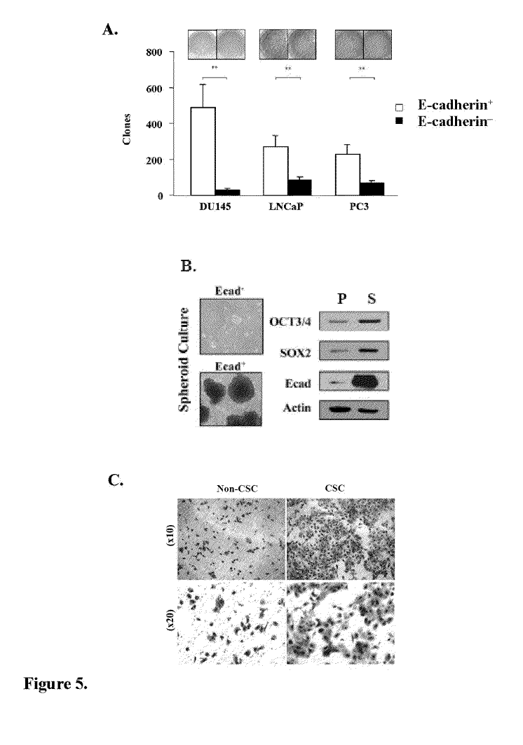

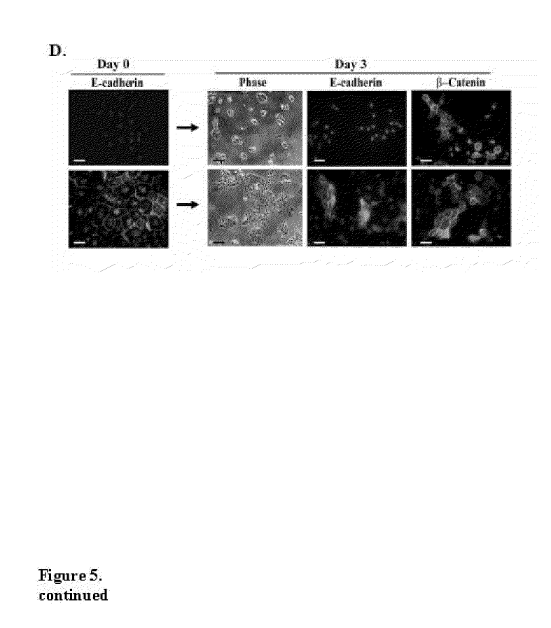

Self-renewal, proliferation, and differentiation are hallmarks of stem cells. To test the clonogenic capacity of isolated stem cells, the prostate tumor stem cells isolated by FACS analysis were cultured in semisolid medium of soft agar for 2-3 weeks until colonies were well-formed. For each cell line, tumor stem cells formed larger and more clones than non-stem tumor cells (P<0.01) (FIG. 5A). This difference was not due to the adhesion properties conferred by E-cadherin in the positive cells, as approximately equal numbers of E-cadherin⁺ and E-cadherin⁻ cells attached upon initial plating. Because both normal and neoplastic prostate stem cells from epithelial origin can be expanded under spheroid culture conditions, sorted E-cadherin⁺ and E-cadherin⁻ DU145 tumor cells were cultured in serum-free medium containing EGF and bFGF under low-attachment conditions in order to favor the proliferation of undifferentiated cells. The results showed that only the E-cadherin+ cells had the ability to form prostate spheroids (FIG. 5B). Western blot analysis of the spheroid culture generated from these E-cadherin+ cells further revealed elevated levels of the stem cell reprogramming factors OCT3/4 and SOX2 as compared to the unsorted parental DU145 cell line (FIG. 5B).

The proliferative capability of the cancer stem cells was also demonstrated (FIG. 5C). Stem and non-stem cells sorted from prostate cancer cell lines were plated and observed. To confirm that prostate stem cell-like tumor cells possess self-renewal capacity, E-cadherin⁺ and E-cadherin⁻ DU 145 cells were evaluated by immunofluorescent analysis using E-cadherin and β-catenin antibodies. The stem cells showed a higher rate of proliferation compared to non-stem cells which displayed little or slow proliferation. Cells grown from the cancer stem cells, which express E-Cadherin, could also differentiate into two populations (E-cad positive and E-cad negative) as observed by immunofluorescence (FIG. 5C). After 3 days in culture, both populations were positive for β-catenin, but the E-cadherin⁻ cells proliferated slowly and remained negative for E-cadherin. In contrast, the E-cadherin⁺ cell population was not only highly proliferative but also produced both E-cadherin+ and E-cadherin⁻ subpopulations, suggesting that asymmetrical division occurred during culture and that the E-cadherin⁺ cell population was enriched with stem cells. A transwell assay was used to observe the invasiveness of cells, in which cells are observed for the ability to migrate from one layer to another through holes in the plates. In the transwell assay, prostate cancer stem cells displayed more migration, thus more invasiveness, compared to non-stem cancer cell (FIG. 5D).

The cell adhesion molecule E-cadherin, one classic marker for epithelial cells, has previously been shown to play an important role maintaining the undifferentiated stage of ES cancer stem cells (Eastham et al. 2007. Cancer Res. 67:11254-11262) and to be down-regulated through the epithelial to mesenchymal transition (EMT) during ES cell differentiation. Interestingly, carcinoma cells utilize a similar mechanism to obtain migratory and invasive capability (Theiry, 2002. Epithelial-mesenchymal transitions in tumour progression. Nat. Rev. Cancer 2:442-454). Although, down regulation of E-cadherin has been thought to be correlated with highly invasive tumors and poor prognosis in prostate cancer, several studies fail to support this notion (Rubin et al., 2001. Hum. Pathol. 32:690-697; Saha et al., 2008. Prostate 68:78-84; Tsukino et al., 2004. Urol. Int. 72:203-207; Yates et al., 2007. Co-culturing human prostate carcinoma cells with hepatocytes leads to increased expression of E-cadherin. Br. I Cancer 96:1246-1252). For example, high expression of E-cadherin was observed in prostate carcinoma bone metastases suggesting the transient nature of EMT (Rubin et al., 2001. Hum. Pathol. 32:690-697; Saha et al., 2008 Prostate 68:78-84; Tsukino et al., 2004. Urol. Int. 72:203-207). Other studies suggest that malignant prostate tumor cells, including the α-catenin deleted PC3 cells, up-regulate E-cadherin upon contact with host cells at the site of metastasis such as liver (Yates et al., 2007. Br. J. Cancer 96:1246-1252) and that the TGF-β induced EMT depletes the stem cell enriched “side population” in breast cancer cells (Yin et al., 2008. Cancer Res. 68:800-807.). Taken together these data suggest that tumor cells only transiently down-regulate E-cadherin for invasion and re-expression of E-cadherin occurs after metastatic seeding (Chafer et al., 2006. Cancer Res. 66:11271-11278). The present findings are consistent with the above evidence that the E-cadherin+ cells in prostate tumor cell lines may have incomplete EMT and represent a stem cell-like subpopulation. The complete EMT cells, the E-cadherin− cells, may eventually lose self-renewal and proliferative capacity. Similar to our findings, others have also reported E-cadherin to be highly expressed among stem cell-enriched holoclonal carcinoma cells (Locke et al., 2005. Cancer Res. 65:8944-8950) and tumor spheres (Lang et al., 2001. Br. J. Cancer 85:590-599).

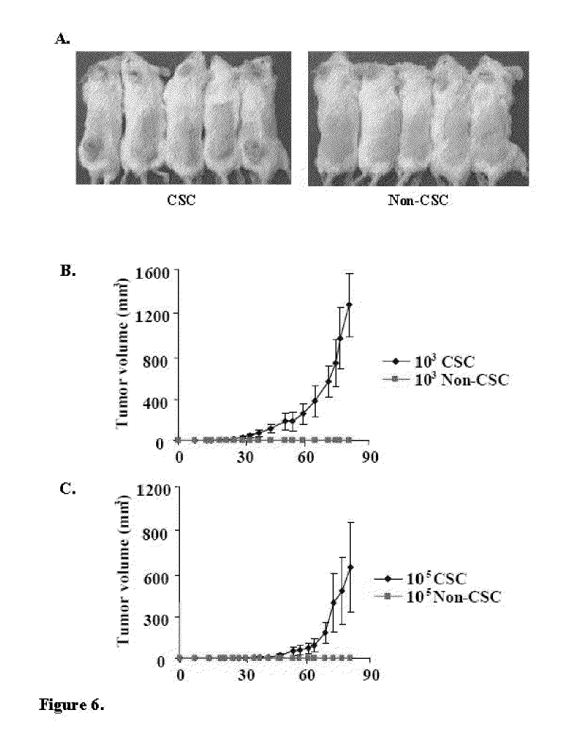

To evaluate the tumorigenic potential of prostate cancer stem cells in vivo, tumor development experiments were performed in male SCID mice using FACS-sorted PC3 and DU145 cancer stem cells. Stem-like or non-stem-like populations (PC3 and DU145) were injected subcutaneously into the mice. Because the two cell lines used have different genetic backgrounds which affect tumor formation, different doses were used in the experiments (1×10³ PC3 cells/mouse, FIG. 6B; and 1×10⁵ DU145 cells/mouse, FIG. 6C). Animals were monitored; and tumor sizes were measured. Mice that were injected with cancer stem cells all developed prostate cancer solid tumors within 30 days after cell inoculation and died within 80 days after cell inoculation (FIGS. 6A-6C). In contrast, mice receiving non-stem cancer cells did not develop tumors during 80 days of observation. These results suggest that the prostate cancer stem cells, isolated and characterized under the conditions described here, possessed higher clonogenic and tumorigenic capacity than non-stem prostate cancer cells and were capable of initiating tumors.

### Example 3

### Metastatic Prostate Tumor-Initiating Cells Expressed Five Transcription Factors Important for the Induction of Pluripotent Stem Cells from Somatic Cells

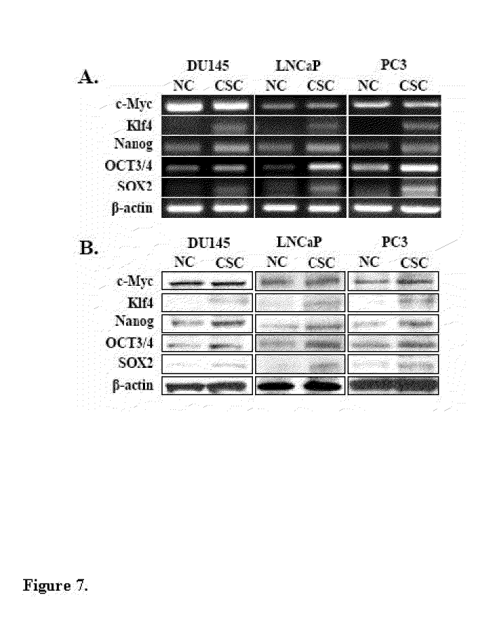

Studies have shown that the embryonic genes, such as OCT3/4 and Nanog, may function in the self-renewal of pluripotent stem cells. Furthermore, a recent study by Yamanaka's group showed that c-Myc, Klf4, Sox2 and OCT3/4 may function in the induction of pluripotent stem cells from somatic cells (Takahashi and Yamanaka, 2006). To determine whether c-Myc, Klf4, Nanog, Sox2 and OCT3/4 play an important role in cancer, the expression of these genes was analyzed by RT-PCR in prostate tumor-initiating cells and non-stem tumor cells purified from the DU145, LNCaP and PC3 cell lines. Increased mRNA levels of Klf4, Nanog, OCT3/4 and Sox2 were observed in the tumor-initiating cells compared to non-stem tumor cells (FIG. 7A). Klf4 and Sox2 expression was enhanced within the tumor-initiating cell populations, compared to non-stem cells, among all three types of metastatic prostate cancer cell lines. High mRNA levels of c-Myc were observed in both cell populations for all prostate cancer cell lines. Western-blotting analysis confirmed similar protein expression patterns for c-Myc, Klf4, Nanog, OCT3/4 and Sox2 (FIG. 7B).

### Example 4

### c-Myc, Klf4, Nanog, OCT3/4 and Sox2 are Expressed In Human Prostate Cancer Tumor Tissue

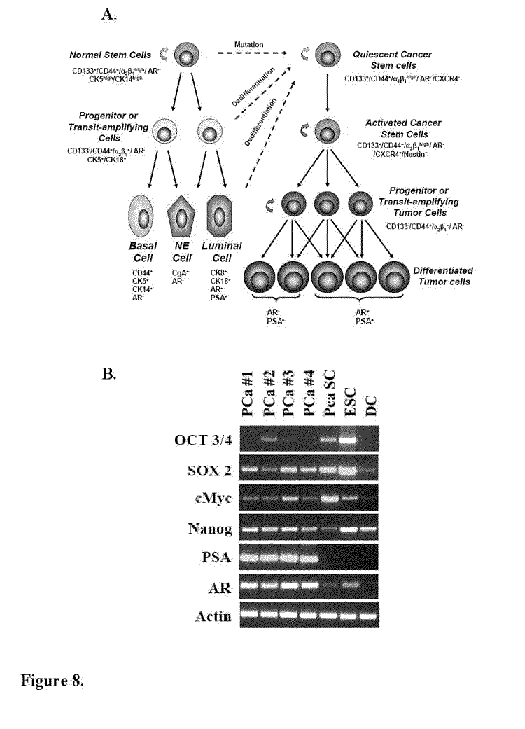

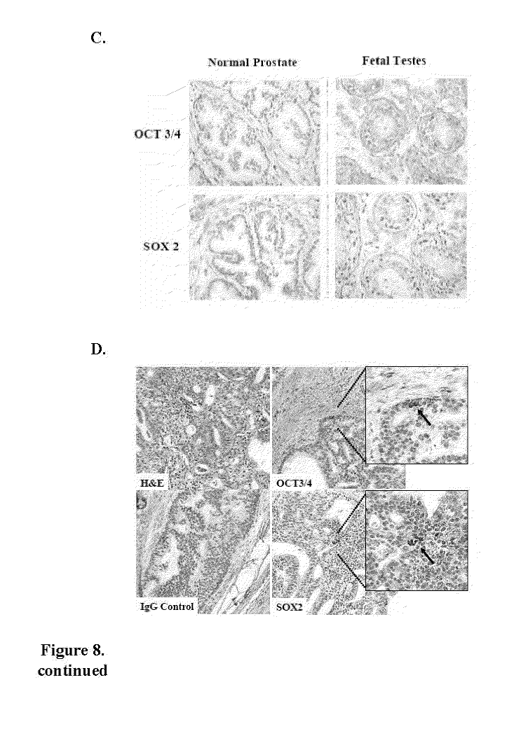

Because the results indicated that a population of stem cells were present in prostate cancer cell lines, human prostate cancer tumor tissue was also examined for the presence of prostate cancer stem cells. Without being bound to any particular theory, prostate neoplasia could arise from the proliferation of prostate cancer stem cells, which arise from the mutation of normal stem cells in the prostate or the de-differentiation of differentiated cells in the prostate (FIG. 8A). Prostate cancer stem cells from tumors would be expected to express the OCT3/4, Sox2, c-Myc, and Nanog markers observed in the prostate cell lines. RT-PCR analysis of four separate tumor tissue samples unenriched for stem cells demonstrated a similar expression profile for the OCT3/4, Sox2, c-Myc, and Nanog markers compared to the isolated stem cells from the prostate cancer cell lines (FIG. 8B). Prostate specific antigen (PSA) and androgen receptor (AR), prostate tissue-specific markers were highly expressed in prostate tumor tissue. As the isolated prostate stem cells are undifferentiated, they were not expected to express PSA. In situ immunohistochemical analysis on prostate tumor tissue revealed cells with high expression of OCT 3/4 and SOX2 in a small population of cells, which were not observed in normal prostate tissue (FIGS. 8C and 8D). These results show that c-Myc, Klf4, Nanog, OCT3/4 and Sox2 markers can be used to identify prostate cancer stem cells and that prostate cancer stem cells are present in prostate tumors.

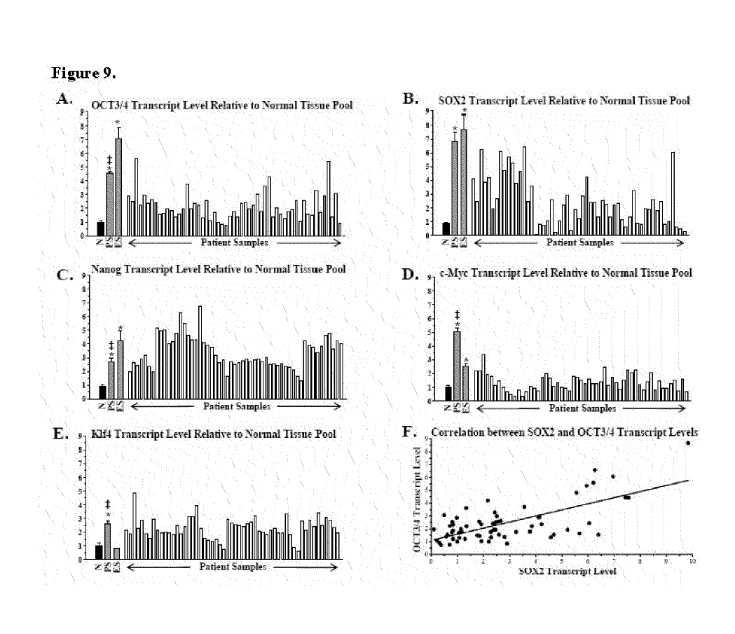

The expression of pluripotent stem cell reprogramming factors in human prostate carcinoma was further examined by RT-PCR analysis of tumor tissue samples from 55 prostate cancer patients and compared to a pooled normal prostate tissue sample from 32 Caucasian males. The hESC line H9 served as a positive control. OCT3/4, SOX2, Nanog, c-Myc, and Klf4 mRNA transcripts for were elevated in more than 50% of the prostate cancer samples compared to the normal prostate tissue pool (FIGS. 9A-9E). Densitometric analysis of mRNA transcripts revealed up to 6-fold activation (after standardizing to normal tissue) of these pluripotent stem cell reprogramming genes in prostate cancer samples compared to the normal prostate tissue pool. The expression pattern of these stem cell reprogramming genes was heterogeneous among patients.

Previously, a primary prostate stem cell-like line was isolated from malignant human tumors that exhibit a stem cell-like phenotype in a neurosphere culture system, and established in vitro under conditions that exploit anchorage independence, serum starvation, and in the presence of pleiotropic growth factors epithelial growth factor (EGF) and basic fibroblast growth factor (bFGF) (Gibbs et al., 2005. Neoplasia 7:967-976). Total RNA from the primary prostate stem cell-like line was extracted for RT-PCR analysis. These primary prostate cancer cells (prostate tumor sphere cells (PS, crosshatch)) were found to have elevated expression of OCT3/4, SOX2, Nanog, c-Myc and Klf4 consistent with a stem cell-like phenotype (FIGS. 9A-9E).

The correlation among the five transcription factors was analyzed using a Newman Keuls multiple comparison test. Both the prostate sphere culture and the ES cell culture were statistically different from the normal tissue pool and also from each other, with two exceptions. In the SOX2 analysis, the prostate sphere culture was not statistically different from the ES cell culture. In the Klf4 analysis, the ES cell culture was not statistically different from the normal tissue pool. Most importantly, in the 55 prostate tissue samples Spearman analysis on all possible combinations of transcription factors demonstrated that significance was reached between OCT3/4 and SOX2 (Spearman correlation coefficient of 0.4730, p<0.0001) suggesting a possible functional link between OCT3/4 and SOX2 in prostate cancer (FIG. 9F).

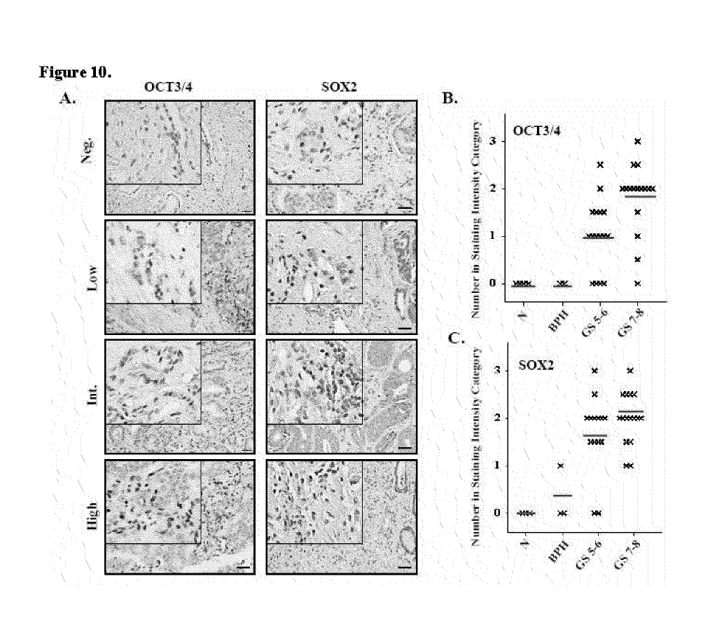

To identify a stem cell-like subpopulation in primary prostate tumor tissue, immunohistochemical staining (FIG. 10A) and analysis of the key stem cell regulators OCT3/4 and SOX2 was performed. The staining intensity of these factors was evaluated using a tissue microarray comprised of two core tissue samples from each of 35 localized prostate tumors (Gleason scores from 5 to 8) as well as 5 benign prostate hyperplasia (BPH) tissues. Nuclear OCT3/4 and SOX2 staining was observed in 76 and 81% of prostate tumor tissues respectively, but not in the BPH samples. The extent of nuclear positive staining in the prostate tumor samples varied widely.

Consequently, the data were stratified into 4 staining categories: negative, low (<5%), intermediate (5-25%), or high (26-50%); none of the samples showed more than 50% nuclear staining. Representative patterns of nuclear staining are shown in FIG. 10A. The number of OCT3/4 or SOX2 expressing cells was significantly lower in the normal prostate and BPH samples as compared to the prostate tumor tissues (FIGS. 10B and 10C). Further, in the prostate tumor tissues samples, increasing numbers of OCT3/4 and SOX2 expressing cells were evident with increasing Gleason scores, suggesting that these cells play a role during prostate cancer progression.

Defined stem cell transcription factors OCT3/4, SOX2, Nanog, c-Myc and Klf4 have been recently reported for reprogramming pluripotent stem cells from differentiated somatic cells (Takahashi and Yamanaka, 2006. Cell 126:663-676; Okita et al., 2007. Nature 448:313-317; Wernig et al., 2007. Nature 448:318-324.). Similar to tumor cells, the transformed or so-called induced pluripotent stem cells (iPS) are immortal, proliferate rapidly and form tumors in immune-deficient mice. As a group, these five transcription factors clearly demonstrate their putative role in transforming adult somatic cells. In the results described herein, these five stem cell transcription factors were expressed not only in the pluripotent stem cells, but also in prostate tumor-initiating cells. Without being bound to any particular theory, the existence of these ES cell genes in both tumor-initiating cells and iPS cells suggest that the expression and distribution of these five factors might be important for determining the fate of these adult stem cell-like cells during the evolution of a normal to a cancerous stem cell.

### Example 5

### Prostate Cancer Stem Cells are Resistant to Conventional Cancer Treatments and are Immune Privileged or Immunosuppressive

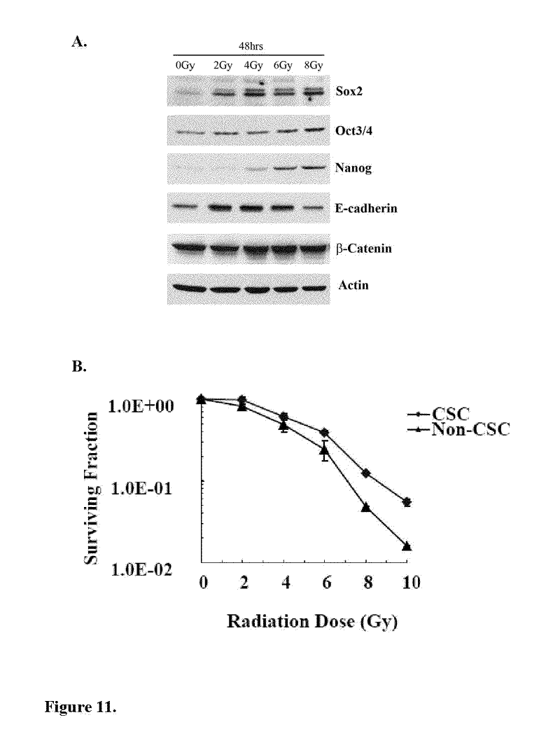

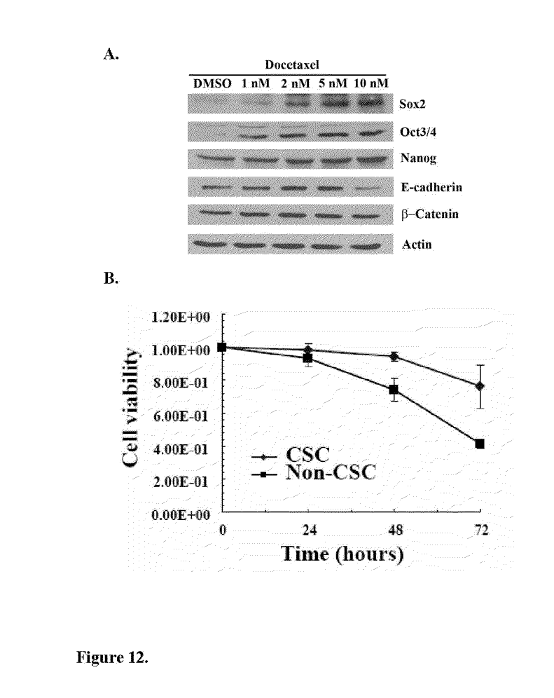

Prostate cancer stem cells from metastatic prostate cancer cell lines were examined for their sensitivity to conventional cancer treatments (e.g., radiation and chemotherapy). Irradiation performed on metastatic prostate cancer stem cell line resulted in the increased detection of Sox2, Oct 3/4 and Nanog expression, possibly due to the enrichment of prostate cancer stem cells with increasing radiation dose (FIG. 11A). When surviving fractions were quantified, prostate cancer stem cells demonstrated more resistance to radiation than non-stem prostate cancer stem cells (FIG. 11B). Metastatic prostate stem cell lines were also treated with Docetaxel, a frontline treatment for drug-resistant cancer cells. Treatment with Docetaxel also resulted in the increased detection of Sox2, Oct 3/4 and Nanog expression with increasing dose (FIG. 12A). Prostate cancer stem cells showed more cell viability compared to non-stem prostate cancer cells, when both cell types were exposed to Docetaxel (FIG. 12B). These results showed that prostate cancer stem cells were resistant to conventional cancer treatments.

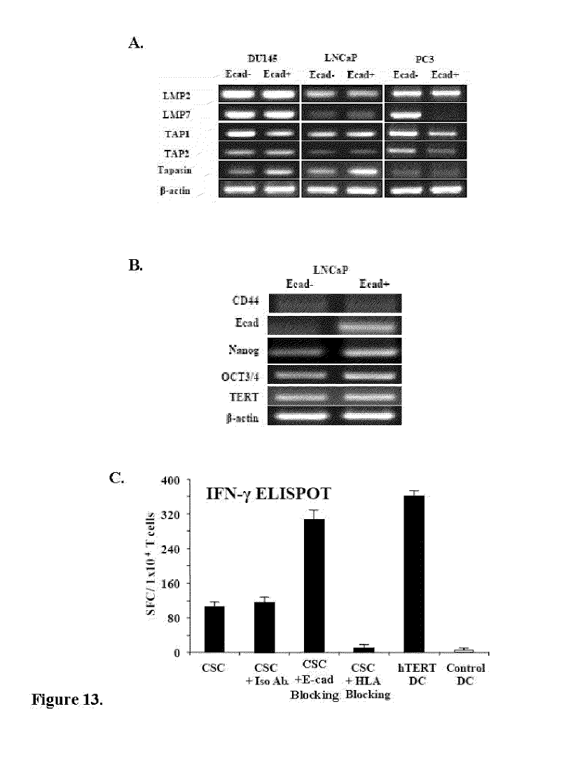

Because the cancer stem cells were relatively refractory to conventional therapies, which are unlikely to be curative and relapses would be expected from prostate cancer stem cells. Prostate cancer stem cells were also studied for treatment using targeted, active immunotherapy (Schuler et al., Curr Opin Immunol. 2003 April; 15(2):138-47, 2003), which employs the cancer stem cell-specific cytotoxic T cells patients own immune system. To explore this possibility, MHC class I antigen presenting pathway in the enriched prostate tumor-initiating cells were screened by RT-PCR analysis. Various defects in expression were observed in stem cells from all three prostate cancer cell lines: DU145 tumor-initiating stem cells have downregulated TAP1 expression; LNCaP tumor-initiating stem cells have low expression of LMP7 and TAP2; and PC3 tumor-initiating cells have little or not expression of LMP7 and TAP2 (FIG. 13A). Thus, the data suggests that genetic defects in the antigen presenting machinery of prostate tumor-initiating stem cells may inhibit antigen presentation in prostate stem cells. These results suggest that prostate cancer stem cells may evade the immune system via defects in the MHC class I antigen presentation pathway.

The ability of T-cells to identify prostate cancer stem cells was also analyzed by IFN-ELISPOT. In the IFN-γ ELISPOT assay, T-cells are mixed with sample cells and the T-cells secrete IFN-γ upon recognition of tumor cells, which is indicated by the detection of IFN-γ within a colony of tumor cells. LNCaP prostate cancer stem cells, which do not express CD44, were used in the IFN-γ ELISPOT assay (FIG. 13B). LNCaP prostate cancer stem cells showed low levels of detection by T-cells, although still higher than when MHC antigens were completely blocked by HLA antibody (FIG. 13C). When cancer stem cells were exposed to E-cadherin blocking antibody, there was a 4-fold increase in the recognition. The difference in the indicates that are able to avoid T-cell detection and are immune privileged or immunosuppressive.

### Example 6

### Disruption of the Stem Cell Transcriptional Balance Resulted in Cell Death in the Metastatic Prostate Tumor-Initiating Cells

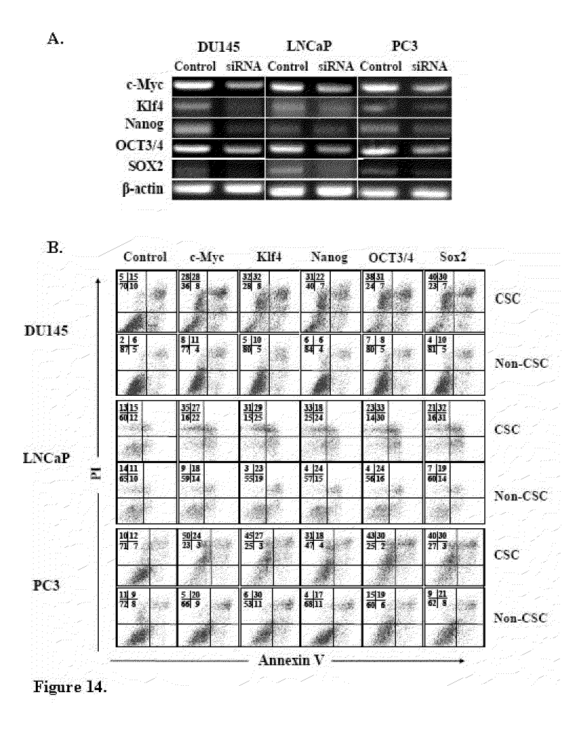

To explore the function of c-Myc, Klf4, Nanog, OCT3/4 and Sox2 stem cell transcription factors, siRNAs specific for these targets were used to inhibit their gene expression. siRNAs specific for targeting c-Myc, Klf4, Nanog, OCT3/4 and Sox2 successfully reduced the expression of the selected genes, as confirmed by RT-PCR analysis, showing the down-regulation of the corresponding genes in the tumor-initiating cells (FIG. 14A).

To examine the stem cell transcriptional balance in the tumor-initiating cells from the metastatic prostate cancer cell lines, tumor-initiating cells and non-stem tumor cells that were purified from DU145, LNCaP and PC3 cells were treated with c-Myc, Klf4, Nanog, OCT3/4 and Sox2 siRNAs separately. Cell death was analyzed using a flow cytometric based annexin V/propidium iodide (PI) binding assay (Lecoeur et al., 2001 Cytometry. 2001 May 1; 44(1):65-72.). siRNAs targeting c-Myc, Klf4, Nanog, OCT3/4 or Sox2 induced cell death in a large percentage of tumor-initiating cells from DU145, LNCaP and PC3 cells (P<0.05, compared with control siRNA) (FIG. 14B). Specifically, after treatment with siRNA for each of the five genes, numbers of live cells (annexin⁻/PI⁻) in the tumor-initiating cell population were significantly reduced. In contrast, annexin V/PI double staining indicated a very low level of cell death in the non-tumor-initiating cells. Each siRNA for c-Myc, Klf4, OCT3/4 or Sox2 induced more than 50% cell death in all three prostate tumor-initiating cell types, especially in the LNCaP cell line where cell death was observed to be more than 70%. The siRNA for Nanog had less impact on cell death when compared to the other four factors. Disruption of the stem cell transcriptional balance induced more annexin⁻/PI⁺ cells in tumor-initiating cells from the DU145 and PC3 lines than the cells from the LNCaP line which had a high percentage of annexin⁺ cells. These results demonstrate that the transcriptional balance of c-Myc, Klf4, Nanog, OCT3/4 and Sox2 is important to the survival of tumor-initiating cells derived from these well-known metastatic prostate tumor lines.

The identification of stem cell-like tumor-initiating cells in prostate cancer models offers tremendous utility in further defining the nature and therapeutic vulnerability of putative prostate cancer stem cells. Data presented here reveal the importance of maintaining transcriptional balance for the survival of tumor-initiating cells. Interruption of this balance, for example, via the change of a single transcription factor, resulted in inhibition of tumor growth in vivo. These findings may have significant implications for identifying new strategies for cancer treatment (Dean et al., 2005. Nat. Rev. Cancer 5:275-284; Diehn et al., 2006. J. Natl. Cancer Inst. 98:1755-1757; Dingli et al., 2006. Stem Cells 24:2603-2610).

### Example 7

### Inhibition of In Vivo Tumorigenicity Using Oct3/4 or Sox2 Short Hairpin RNAs (shRNA)

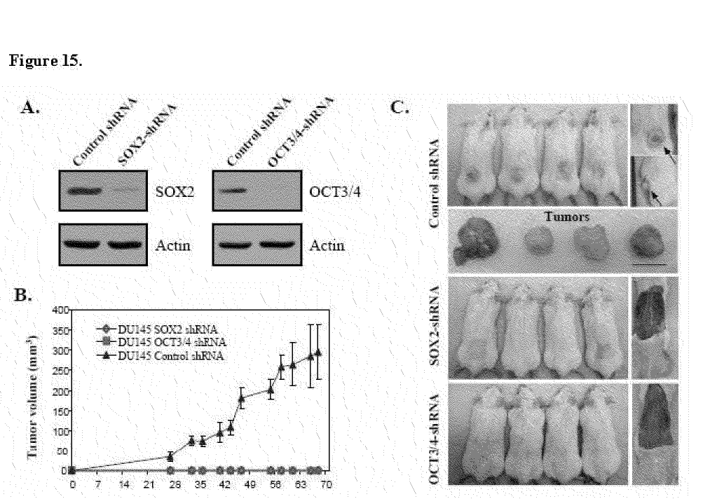

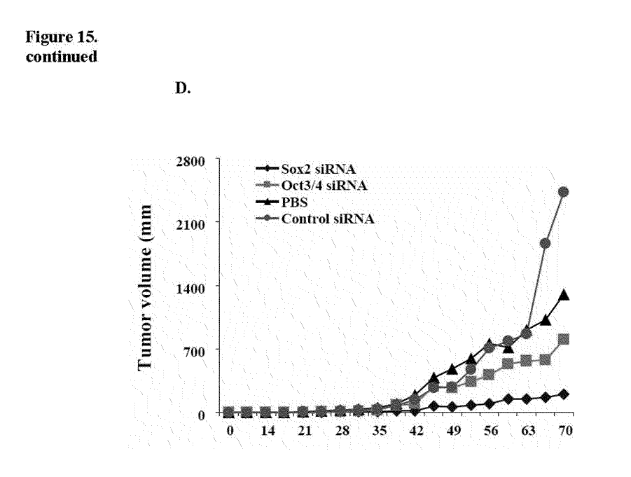

To gain further insights into the importance of stem-cell transcription factors in tumorigenicity, DU145 prostate cancer cells were infected with plasmids encoding shRNAs targeting OCT3/4 or Sox2 or with shRNA control plasmids. The effect of inhibiting OCT3/4 and Sox2 in prostate cancer stem cells was examined in the SCID mouse model of tumorigenicity. Prostate cancer stem cells were pre-treated with siRNAs or shRNAs, before being subcutaneously injected into mice. Both OCT3/4 and SOX2 shRNA sequences individually dramatically reduced the expression of their respective protein (FIG. 15A). Equal numbers of OCT3/4 shRNA, Sox2 shRNA or control shRNA-transfected DU145 cells then were inoculated into SCID mice and tumor growth was monitored. Mice injected with prostate cancer stem cells pre-treated with either OCT3/4 shRNA or Sox2 shRNA failed to develop detectable tumors over an observation period of 10 weeks (FIGS. 15B and 15C). In contrast, cells infected with control shRNA plasmids developed detectable tumor growth (4 out of 5 mice) within 3 weeks of cell inoculation. Mice receiving prostate cancer stem cells pre-treated with OCT3/4 and Sox2 siRNAs had smaller tumors than the control treated prostate cancer stem cells (FIG. 15D). When used in combination, siRNAs and shRNAs for OCT3/4 and Sox2 would have a greater effect on the reduction of tumor size. The results show that the transcriptional balance of OCT3/4 and Sox2 is important to the tumorigenicity of prostate stem cells.

### Example 8

### OCT 3/4, Nanog, and E-cadherin are Expressed in Cancer Stem Cells in Human and Mouse Cancer Cell Lines and Tumors

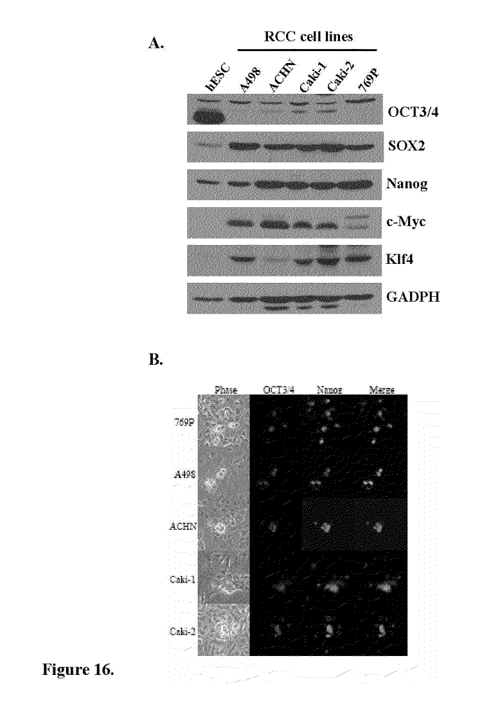

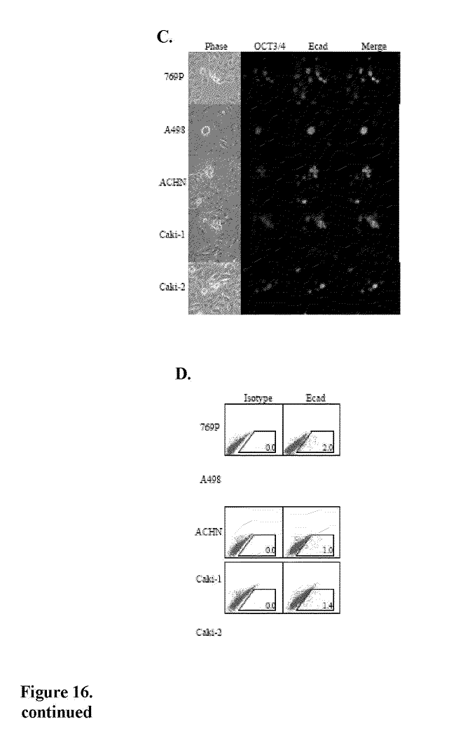

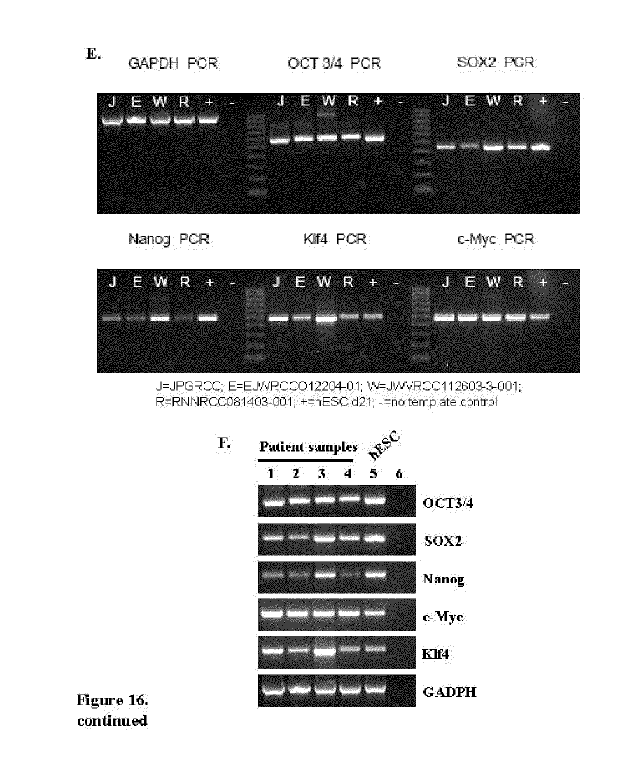

To examine whether OCT3/4 and Nanog were expressed as proteins by putative cancer stem cells in other tumor cell lines, cultured cancer cells and tumor tissue were examined for OCT3/4 and Nanog expression by Western blot analysis and immunofluorescence staining. In the five renal cancer cell lines examined (769P, A498, ACHN, Caki-1, and Caki-2), expression of OCT3/4, SOX2, Nanog, c-Myc, and Klf4 was detected by Western blot analysis (FIG. 16A). Human renal cancer cell lines contained a distinct population of cells that displayed high expression of both OCT3/4 and Nanog by immunofluorescence microscopy (FIG. 16A). These cells were readily observed in all five renal cancer cell lines examined (769P, A498, ACHN, Caki-1, and Caki-2). These results suggest the existence of a population of cancer stem cells in 769P, A498, ACHN, Caki-1, and Caki-2 renal cancer cell lines. The cultured renal cancer cells were also examined for the co-expression of OCT3/4 and E-cadherin by immunofluorescence microscopy. In all renal cell lines examined, OCT3/4 and E-cadherin showed co-expression in a population of cells (FIG. 16B). In three renal cell lines examined, E-cadherin+ cells represented a small population (769P—2.0%, ACHN—1.0%, Caki-1—1.4%) that was distinguishable by flow cytometry (FIG. 16C) and that corresponded to renal cancer stem cells. These results demonstrate that renal cancer stem cells can be isolated from renal cancer cell lines using E-cadherin as a marker.

Renal cancer stem cells from primary tumors would be expected to express the OCT3/4, Sox2, c-Myc, and Nanog markers observed in the renal cancer cell lines. In two exemplary experiments, RT-PCR analysis of four separate primary tumor tissue samples unenriched for stem cells demonstrated a similar expression profile for the OCT3/4, Sox2, Nanog, Klf4, and c-Myc markers compared to human embryonic stem cells (FIGS. 16E and 16F). These results demonstrate that OCT3/4, Sox2, c-Myc, and Nanog markers are likely present in multiple types of mammalian cancers.

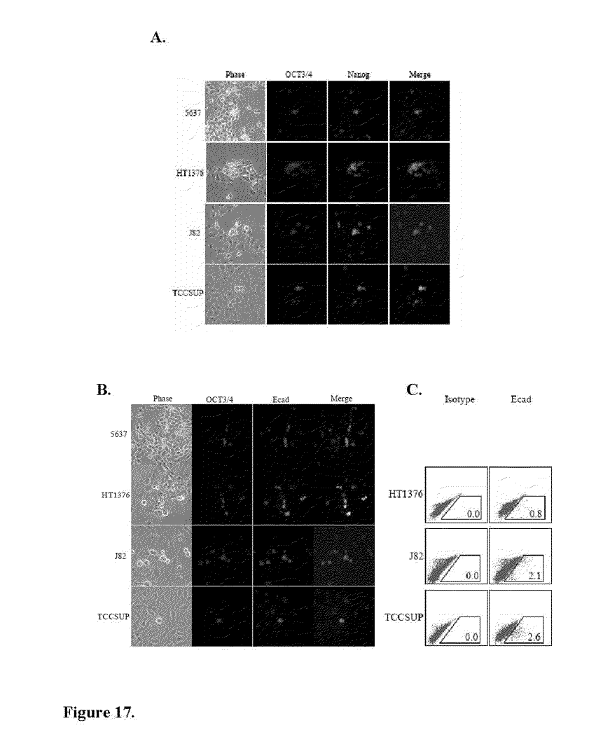

Human bladder cancer cell lines were also examined for the presence of bladder cancer stem cells. A distinct population of bladder cancer cells displayed high expression of both OCT3/4 and Nanog by immunofluorescence microscopy and were readily observed in all four bladder cell lines examined (5637, HT1376, J82, and TCCSUP) (FIG. 17A). These results suggest the existence of a population of cancer stem cells in 5637, HT1376, J82, and TCCSUP bladder cancer cell lines. The cultured bladder cancer cells were also examined for the co-expression of OCT3/4 and E-cadherin by immunofluorescence microscopy. In all bladder cell lines examined, OCT3/4 and E-cadherin showed co-expression in a population of cells (FIG. 17B). In three bladder cell lines examined, E-cadherin+ cells represented a small population (HT1376—0.8%, J82—2.1%, TCCSUP—2.6%) that was distinguishable by flow cytometry and that corresponded to bladder cancer stem cells. These results demonstrate that bladder cancer stem cells can be isolated from bladder cancer cell lines using E-cadherin as a marker.

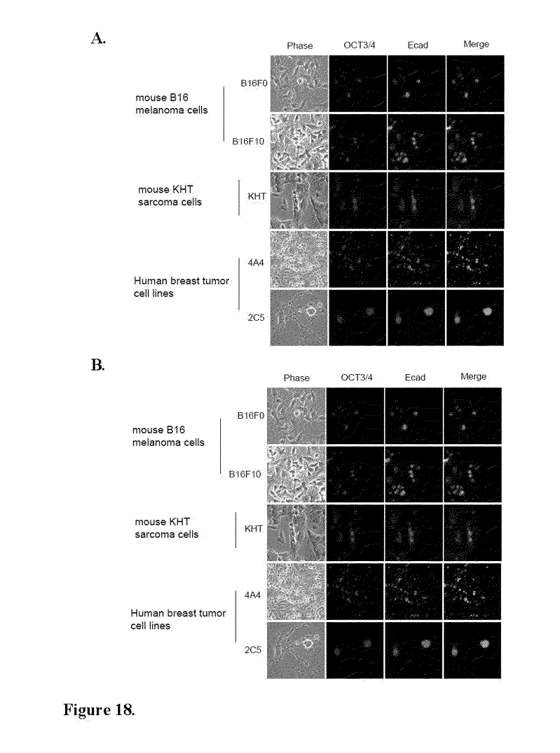

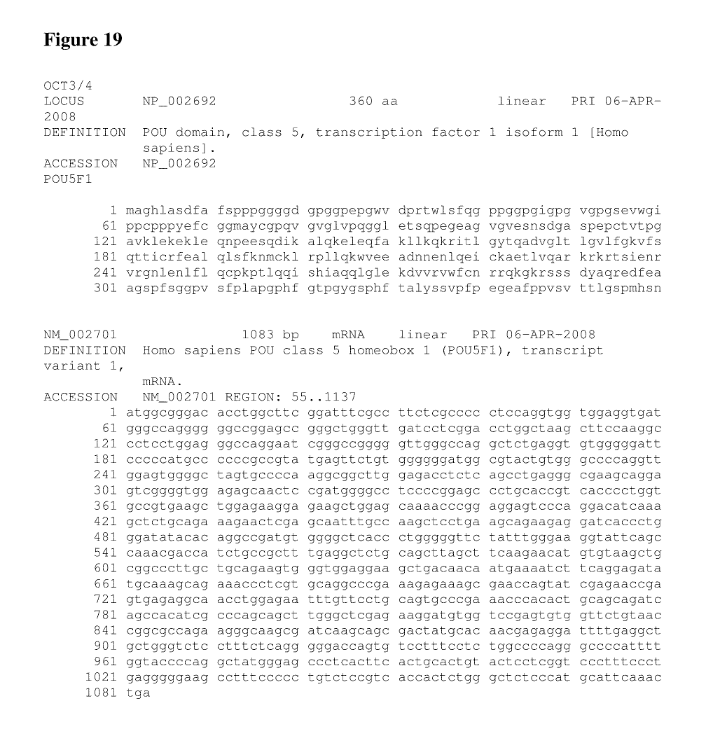

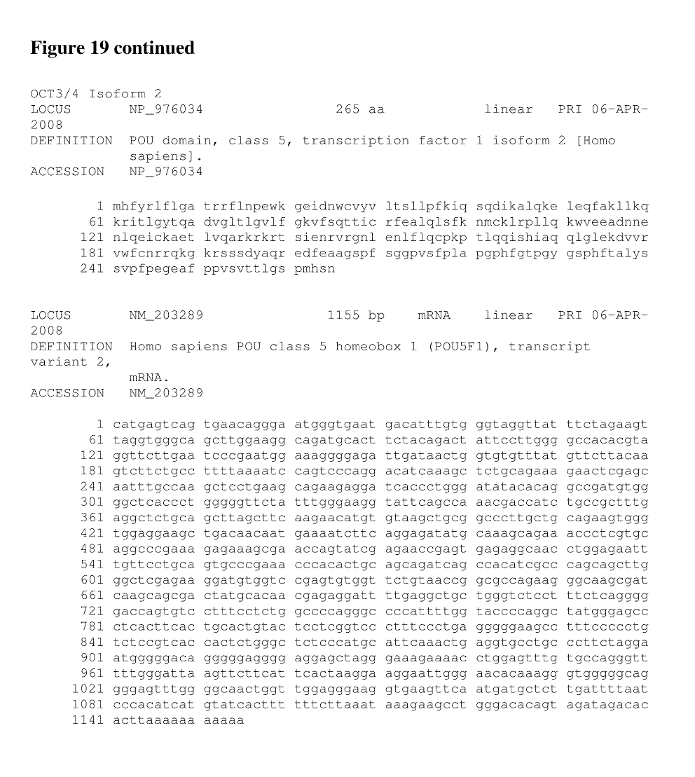

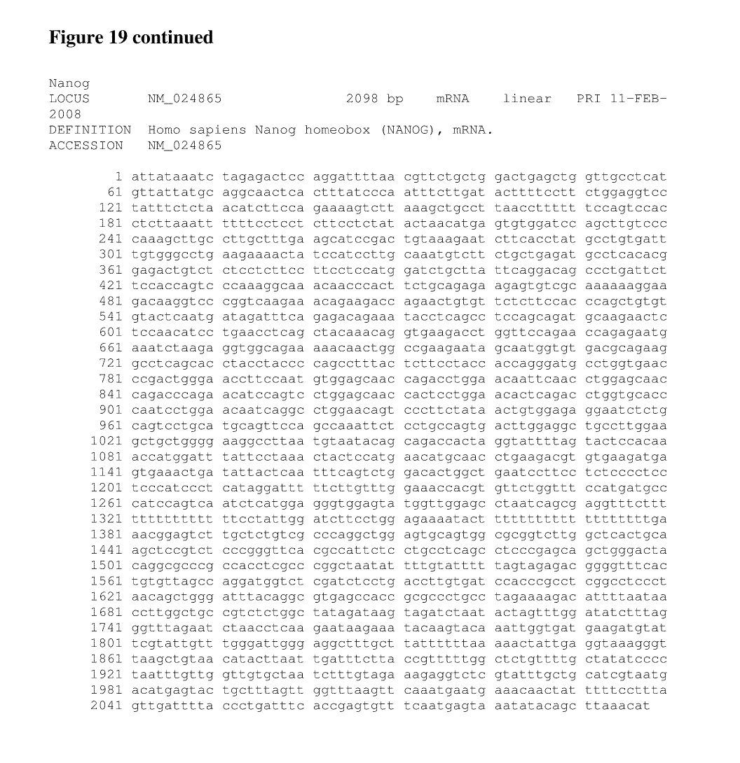

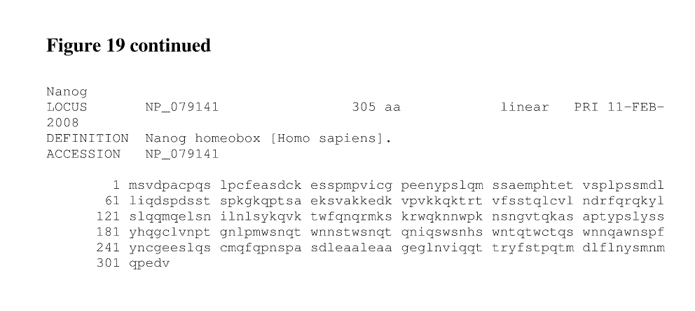

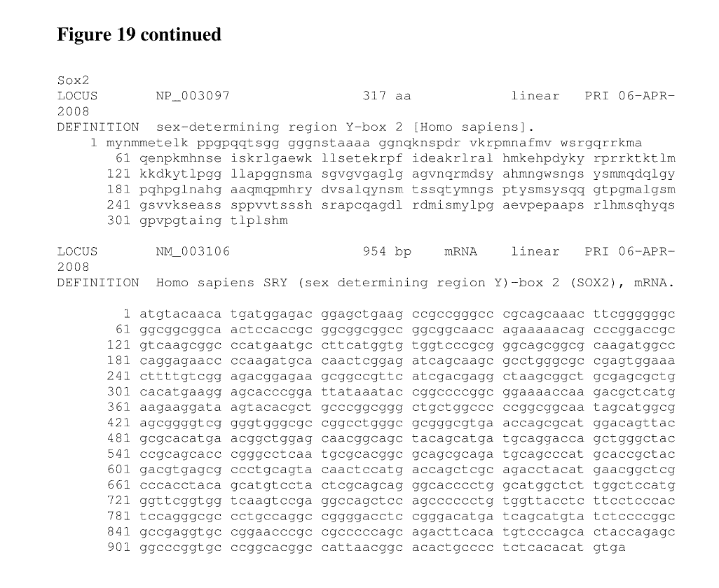

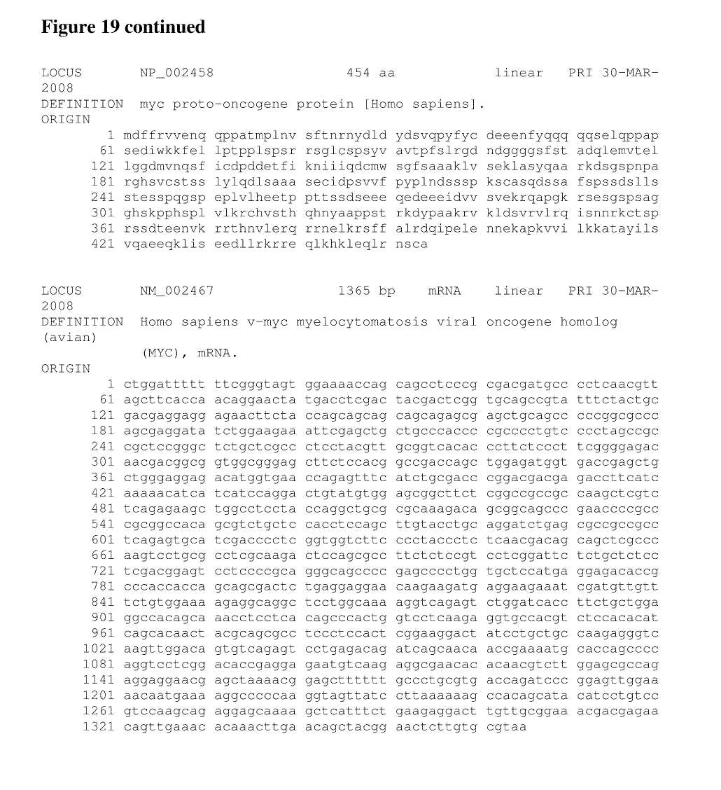

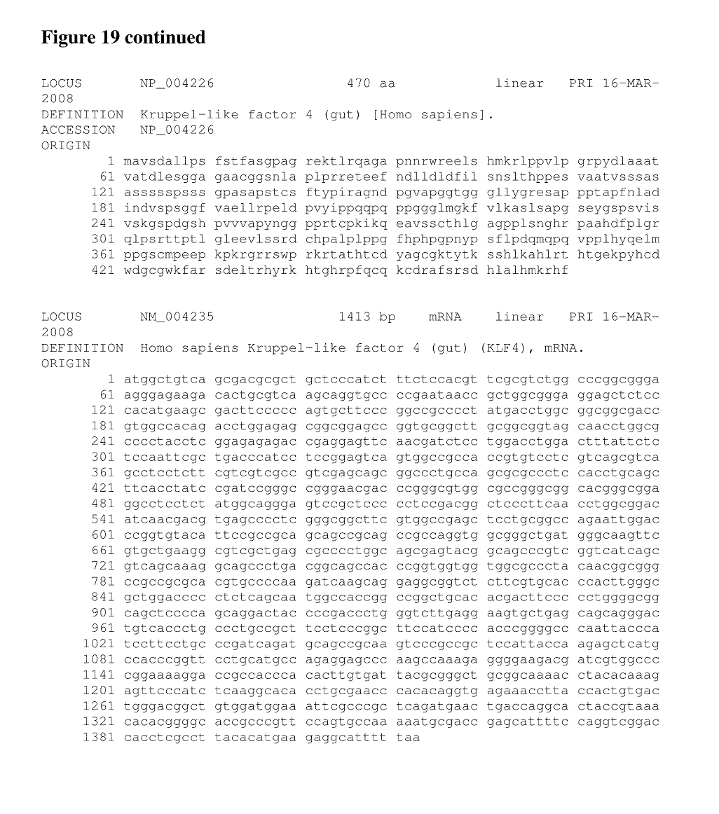

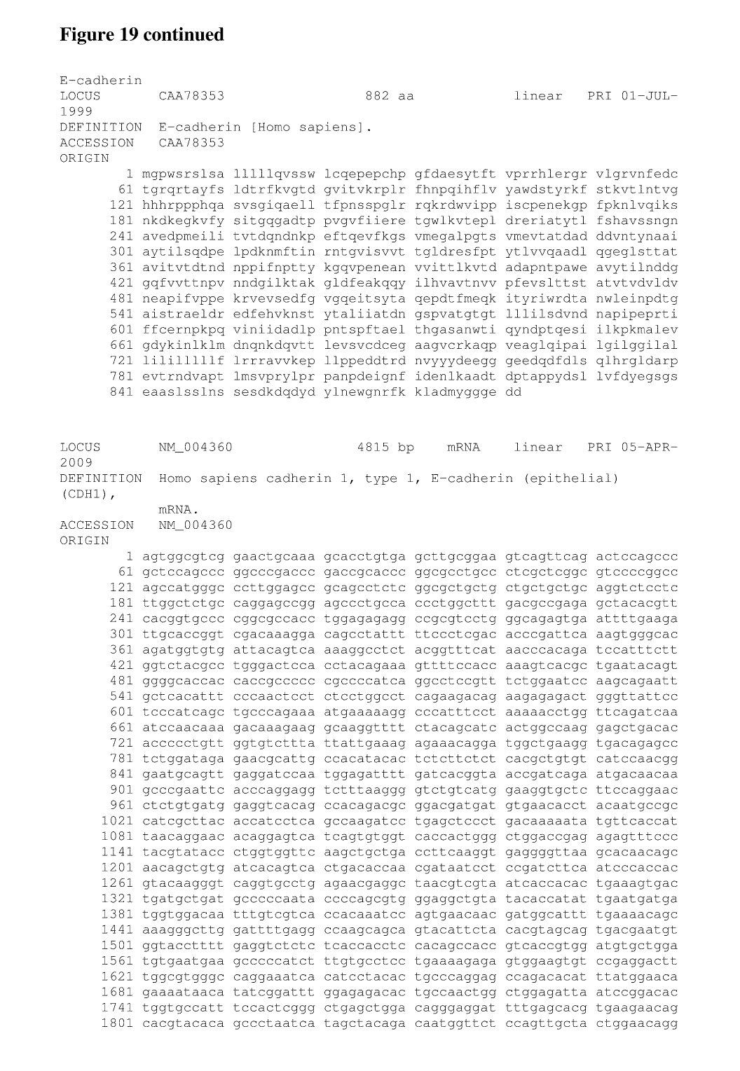

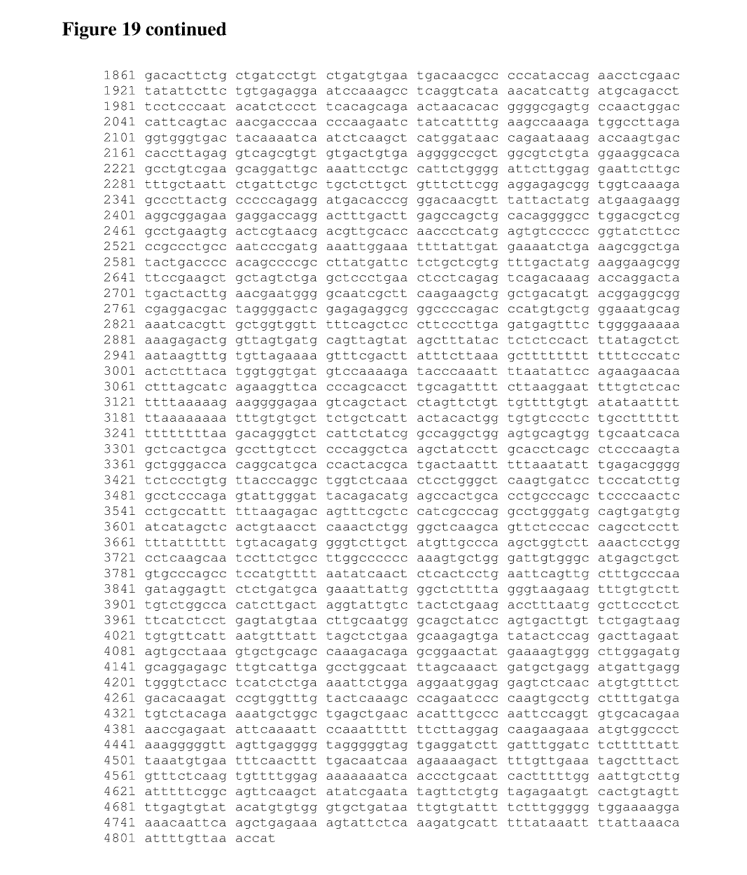

Examination of human breast tumor cell lines 4A4 and 2C5 by immunofluorescence microscopy also showed high expression of the stem cell markers OCT3/4 and Nanog in breast tumor cells (FIG. 18A). Because the cell surface marker E-cadherin was also expressed in the breast tumor stem cells expressing OCT3/4 (FIG. 18B), it would be possible to isolate breast tumor stem cells by using E-cadherin as a marker. Mouse tumor cells were also examined for the stem cell markers OCT3/4 and Nanog. In B16F0 and B16F10 mouse B16 melanoma cells and mouse KHT sarcoma cells, OCT3/4 and Nanog were co-expressed (FIG. 18A). E-cadherin was also expressed in the cells expressing OCT3/4 (FIG. 18B) and could also be used as a cell surface marker to isolate stem cells. These results demonstrate that cancer stem cells are present in tumors or cancer cell populations, represent a small, but distinct population of cancer cells in all types of cancers, and are expected to be present in cancers in mammalian species.

The results reported above were obtained using the following methods and materials.

### Human Embryonic and Prostate Cancer (PC) Cell Lines

Human metastatic prostate cancer cell lines were used in the studies described herein: DU145 (established from brain metastasis), LNCaP (established from lymph node metastasis) and PC3 (established from bone metastasis). The human prostate cancer cell lines were obtained from the American Type Culture Collection (ATCC; Manassas, Va.). Cells were grown in appropriate growth medium (ATCC) as suggested by ATCC. The human embryonic stem cell line H9 was obtained from the National Stem Cell Bank and was cultured as described in Su et al. (Differentiation of human embryonic stem cells into immunostimulatory dendritic cells under feeder-free culture conditions. 2008. Clin. Cancer Res. 14:6207-6217). Clinical diagnoses were confirmed by the Department of Pathology at the University of Florida. Human prostate cancer tissue microarrays were purchased from Cybrdi.

### Reagents

Commercially available PE-conjugated or FITC-conjugated monoclonal Abs (mAbs) against human CD9, CD24, CD44, E-cadherin, and mouse IgG1 isotype control were used in the experiments described above (BD PharMingen; San Diego, Calif.). Commercially available FITC-conjugated annexin V was used in the experiments described above (BD PharMingen). Commercially available PE-conjugated mAbs against CD133 were used in the experiments described above (Miltenyi Biotech; Auburn, Calif.). Commercially available PE-conjugated or FITC-conjugated Abs against PODXL, SSEA1, and SSEA4 were used in the experiments described above (R&D Systems; Minneapolis, Minn.). Commercially available primary rabbit or mouse Abs against β-actin, c-Myc, Klf4, Nanog, OCT3/4 and Sox2 were used in the experiments described above (Santa Cruz Biotechnology; Santa Cruz, Calif.). Commercially available Agar Noble used in the experiments described above was obtained from Becton, Dickinson and Company (Sparks, Md.). Propidium iodide (PI) and crystal violet were obtained from SIGMA (St. Louis, Mo.).

### Immunofluorescence

For immunofluorescence in the experiments described above, cells were seeded on uncoated glass slides at approximately 2000 cells cm² and cultured for 4 days; cells were fixed at −20° C. in cold methanol for 8 minutes and subsequently washed in phosphate-buffered saline (PBS). Enzyme treatment was not performed. Cells were stained with specific Abs. Non-specific binding of the secondary Abs was reduced with an appropriate serum block. After staining, all slides were examined and pictures were taken using a commercially available fluorescence microscope (Carl Zeiss, Jena, Germany).

Alternatively, cells were grown on glass coverslips, fixed in 4% paraformaldehyde (Sigma), and permeabilized with 0.2% Triton X-100/PBS. The cells were blocked with 10% goat serum/0.05% Triton X-100/PBS before incubating with commercially available primary antibodies (anti-E-cadherin, anti-OCT3/4 from Santa Cruz, anti-SOX2 from AbCam, anti-β-catenin from BD Bioscience) overnight at 4° C. The slides were washed, incubated with commercially available Alexa Fluor 594—and/or Alexa Fluor 488—conjugated secondary antibodies (Molecular Probes) and mounted using a commerically available mounting medium (Vectashield; Vector Laboratories) containing DAPI to counterstain nuclei. The processed cells were examined using a commercially available fluorescence microscope (Zeiss Axiophot microscope).

### Flow-Cytometry Analysis and Fluorescence-Activated Cell Sorting

Flow-cytometry in the experiments described above was performed by standard methods. Flow-cytometry was used to analyze the expression of cell surface molecules. Single cell suspensions were prepared by trypsinization and then incubated in fresh medium on a rocker platform to enable regeneration of cell adhesion molecules. The cells were washed, suspended in PBS containing 1% BSA and 1 mM CaCl2, and stained with commercially available primary antibodies for E-cadherin, PODXL, SSEA1, SSEA4 (R&D Systems), CD44, CD9, CD24, Integrin-α2β1 (BD Pharmingen), ESA (Biomeda), and CD133 (Miltenyi Biotech). In addition, cell death was analyzed using FITC-conjugated annexin V and propidium iodide (PI).

Analyses of fluorescence staining were performed using a commercially available flow cytometer (Becton Dickinson FACScan; San Jose, Calif.). Cells stained with propidium iodide (Sigma) were sorted using a commercially available Fluorescent-activated cell sorting (FACS) system (FACSCalibur flow cytometer, Becton Dickinson). E-cadherin positive and negative cells were sorted by Fluorescent-activated cell sorting (FACS) analysis (Mo-Flo Cell Sorter; Becton-Dickinson). Live single cells were gated for analysis and sorted (FACSAriaSORP Cell Sorter with Diva 6.1 software, Becton Dickinson).

### Prostate Spheroid Culture

The prostate spheroid culture assay was performed according to the method of Shi et al. (Anchorage-independent culture maintains prostate stem cells. 2007. Dev. Biol. 312:396-406).

### Soft Agar Assay

Cancer stem cells and non-cancer stem cells were isolated from DU145, LNCaP and PC3 cells. Cells were suspended in growth medium containing 0.3% agar and layered over a 0.6% agar base layer to a final cell density of 2×10³ cells/well. Cells were fed with fresh growth media every 4-5 days for 2-3 weeks until the colonies were well formed. Clones were stained with 0.005% crystal violet for visualization.

### Reverse Transcription Polymerase Chain Reaction (RT-PCR)

For the experiments described above, total RNA was extracted by using a commercially available kit (RNeasy Mini Kit; Qiagen, Valencia, Calif.), according to the manufacturer's instructions. Reverse transcription reactions were performed by standard methods using a commercially available kit (Transcriptor First Strand cDNA Synthesis Kit; Roche, Indianapolis, Ind.). PCR was performed by standard methods using a commercially available Taq DNA polymerase (Roche) or commercially available PCR mix (GoTaq Green Master Mix, Promega). For the reactions, β-Actin transcript levels were used to normalize the amount of cDNA in each sample. For the experiments described above, primer sets used for RT-PCR analyses are listed in Table 1.

Semi-quantitative RT-PCR was also performed by using a panel of first-strand cDNAs from 48 human prostate samples (TissueScan Prostate Cancer II, Origene). H9 cells, and the primary prostate stem-cell like line were analyzed and normal human prostate RNA (Clontech) was used as a control. Relative transcript levels were normalized to β-actin levels for each case. In order to detect pseudogenes of Oct4A, the procedure of Panagopoulos et. al. 2008. Genes Chromosomes Cancer 47:521-529) was used.

TABLE 1
List of primers for RT-PCR.

| | | product | annealing |
|---|---|---|---|
| Gene name | primer sequence | size | temperature |
| GAPDH | F5′-TGAAGGTCGGAGTCAACGGATTTGGT-3′ | 892 | 60° C. |
| | R5′-CATGTGGGCCATGAGGTCCACCAC-3′ | | |
| B-actin | F5′-CTCTTCCAGCCTTCCTTCCT-3′ | 311 | 55° C. |
| | R5′-TCGTCATACTCCTGCTTGCT-3′ | | |
| B-actin | F5′-CAGCCATGTACGTTGCTATCCAGG-3′ | 140 | 55° C. |
| | R5′-AGGTCCAGACGCAGGATGGCATG-3′ | | |
| Oct3/4 | F5′-ATTCAGCCAAACGACCATCT-3′ | 371 | 55° C. |
| | R5′-CAGCAGCCTCAAAATCCTCT-3′ | | |
| Oct3/4 (4A) | F5′-ACACCTGGCTTCGGATTTCGCCT-3′ | 624 | 60° C. |
| | R5′-GCTTCCTCCACCCACTTCTGCAGC-3′ | | |
| Sox2 | F5′-CCCCCGGCGGCAATAGCA-3′ | 448 | 55° C. |
| | R5′-TCGGCGCCGGGGAGATACAT-3′ | | |
| cMyc | F5′-TACCCTCTCAACGACAGCAG-3′ | 468 | 55° C. |
| | R5′-TCTTGACATTCTCCTCGGTG-3′ | | |
| Nanog | F5′-TCTCCTCTTCCCTCCTCCAT-3′ | 487 | 55° C. |
| | R5′-GGATGTTCTGGGTCTGGTTG-3′ | | |
| K1f4 | F5′-GAGAGAGACCGAGGAGTTCA-3′ | 480 | 55° C. |
| | R5′-CCTTTGCTGACGCTGATGAC-3′ | | |
| K1f4#2 | F5′-CAGCGACGCGCTGCTC-3′ | 987 | 62° C. |
| | R5′-TGCAGGAACCGGGTGGCATG-3′ | | |
| PSA | F5′-GGTGACCAAGTTCATGCTGTG-3′ | 195 | 60° C. |
| | R5′-GTGTCCTTGATCCACTTCCG-3′ | | |
| AR | F5′-GAAGCCATTGAGCCAGGTGT-3′ | 164 | 60° C. |
| | R5′-TCGTCCACGTGTAAGTTGCG-3′ | | |
| B-catenin | F5′-ACTGGCAGCAACAGTCTTACC-3′ | 836 | 60° C. |
| | R5′-TCGTCCACGTGTAAGTTGCG-3′ | | |
| hE-cadherin | F5′-GAACGCATTGCCACATACACT-3′ | 745 | 60° C. |
| | R5′-CTGTGGAGGTGGTGAGAGAGA-3′ | | |
| tert | F5′-GCACGGCTTTTGTTCAGATG-3′ | 407 | 55° C. |
| | R5′-GTTCTTGGCTTTCAGGATGG-3′ | | |

### Histological and Immunohistochemical Analysis

For the experiments described above, commercially available tissue arrays (Cybrdi) were used. Additional tissues were obtained from the Department of Urology at the University of Florida. These were processed as described in Gibbs et al. (Stem-like cells in bone sarcomas: implications for tumorigenesis. 2005. Neoplasia 7:967-976) using either a commercially available OCT3/4 (AbCam) antibody or a commercially available SOX2 (R&D Systems) antibody. To assess nuclear staining, an arbitrary system was used by a pathologist blinded to sample identity. Twenty random fields were examined and the overall percentage of positive nuclear staining was histologically scored.

### Western-Blot Analysis

For the experiments described above, Western-blot analysis was performed by standard methods on isolated cancer stem cells and non-cancer stem cells. Whole cell lysates were prepared in lysis buffer with a commercially available protease inhibitor cocktail (Pierce) at 4° C., followed by centrifugation at 13,000×g for 10 min. Extracts were separated by SDS/PAGE and transferred to nitrocellulose membranes. The membranes were blocked with 5% nonfat dry milk in 20 mM Tris-HCl (pH 7.5), 500 mM sodium chloride, and 0.05% Tween 20 for 2 h and then incubated with commercially available primary antibodies against OCT3/4, c-Myc, β-actin (Cell Signaling), SOX2, Klf4 (Santa Cruz), Nanog (BioLegend), or E-cadherin (BD Bioscience) in the same buffer with 1% BSA (fraction V). After washing, the blots were incubated with an HRP-conjugated secondary antibody and visualized with a commercially available chemiluminescence detection system (Amersham). β-actin-specific Abs were used to ensure equal protein loading.

### Mouse Xenograft Model of Human PC

For the experiments described above, male C.B-17/IcrHsd SCID mice, 5 to 6 weeks old, were obtained from Harlan Sprague Dawley (Indianapolis, Ind.). To examine tumorigenicity, sorted cells from PC3 and DU145 cells were injected subcutaneously into groups of mice (5 mice per group) at a dose of 1×10³ PC3 cells/mouse or 1×10⁵ DU145 cells/mouse; and experiments repeated twice. Experiments were performed under an approved protocol of the Institutional Animal Care and Use Committee of the University of Florida. Animals were monitored, and tumor size was measured twice a week. Mice were humanely sacrificed when moribund or when subcutaneous tumors reached 15 mm in diameter.

siRNA and shRNA Transfection

For the experiments described above, commercially available c-Myc, Klf4, Nanog, OCT3/4, Sox2 and control siRNAs, transfection reagent, and transfection medium (Santa Cruz Biotechnology) were used. Gene silencing of specific target genes was performed according to the manufacturer's protocol. Control siRNA was also used for these experiments.

For shRNA knock-down experiments, commercially available plasmid vectors encoding either OCT3/4 or SOX2 were used (Origene). For transfection, 1.5×10⁵ DU145 cells/well were seeded in 6 well plates in media without antibiotics the day before the experiment. Cells were washed with buffer (Optimem, Invitrogen) and then transfected using a commercially available transfection reagent (Lipofectamine, Invitrogen). Transfected cells were selected using puromycin, pooled, and single-cell cloned before Western blot analysis for OCT3/4 or SOX2 expression.

Student t test was used for the comparison of various experimental groups. Significance was set at P<0.05. One-way ANOVA, Newman Keuls testing and Spearman coefficient of rank correlation were calculated using Prism version 4 (GraphPad Software, Inc.). Significance was set at p<0.05.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for reducing the survival of a neoplastic cell or inducing cell death in a neoplastic cell, the method comprising contacting the cell with an agent that reduces the expression of one or more of OCT3/4, Nanog, Sox2, c-Myc or Klf4 nucleic acid molecule or polypeptide.

2. The method of claim 1, wherein the neoplastic cell is derived from a tissue selected from the group consisting of prostate tissue, renal tissue, bladder tissue, breast tissue, skin, and connective tissue.

3. (canceled)

4. The method of claim 1, wherein the agent is an antibody that specifically binds an OCT3/4, Nanog, Sox2, c-Myc or Klf4 polypeptide, or an inhibitory nucleic acid molecule at least a portion of which is complementary to an OCT3/4, Nanog, Sox2, c-Myc or Klf4 nucleic acid molecule, wherein the inhibitory nucleic acid molecule is an antisense molecule, shRNA, or siRNA.

5. (canceled)

6. (canceled)

7. A method for treating or preventing neoplasia in a subject in need thereof, the method comprising contacting a neoplastic cell in the subject with an agent that reduces the expression of one or more of OCT3/4, Nanog, Sox2, c-Myc and Klf4 nucleic acid molecule or polypeptide.

8. The method of claim 7, wherein the agent is an antibody that specifically binds a OCT3/4, Nanog, Sox2, c-Myc or Klf4 polypeptide or, an inhibitory nucleic acid molecule at least a portion of which is complementary to an OCT3/4, Nanog, Sox2, c-Myc or Klf4 nucleic acid molecule, wherein the inhibitory nucleic acid molecule is an antisense molecule, shRNA, or siRNA.

9. (canceled)

10. (canceled)

11. The method of claim 10, wherein the neoplastic cell is derived from a tissue selected from the group consisting of prostate tissue, renal tissue, bladder tissue, breast tissue, skin, and connective tissue.

12. A method for identifying a neoplasia in a subject, the method comprising identifying an increased level of a nucleic acid molecule or polypeptide Marker selected from the group consisting of OCT3/4, Nanog, Sox2, c-Myc and Klf4 in a biological sample derived from the subject, relative to the level present in a reference, thereby identifying the subject as having neoplasia.

13. A method for identifying a metastatic neoplasia or a neoplasia having a propensity to metastasize, the method comprising comparing the level of a nucleic acid molecule or polypeptide Marker selected from the group consisting of OCT3/4, Nanog, Sox2, c-Myc and Klf4 in a biological sample, relative to the level present in a reference, wherein an increase in the level of one or more of said Markers identifies the neoplasia as metastatic or as having a propensity to metastasize, and wherein the absence of an increase in the level of one or more Markers identifies the neoplasia as non-metastatic or as lacking the propensity to metastasize.

14-16. (canceled)

17. A method of determining the prognosis of a subject having neoplasia, the method comprising determining the level of one or more of a nucleic acid molecule or polypeptide Marker selected from the group consisting of OCT3/4, Nanog, Sox2, c-Myc and Klf4 in a biological sample derived from the subject, relative to the level present in a reference, wherein an increase in the level of each of said Markers identifies the subject as having a poor prognosis, and wherein the absence of alteration in the level of one or more of said Markers identifies the subject as having a good prognosis.

18. (canceled)

19. (canceled)

20. A method of selecting an appropriate therapy for a subject having neoplasia, the method comprising comparing the level of a Marker selected from the group consisting of OCT3/4, Nanog, Sox2, c-Myc and Klf4 nucleic acid molecule or polypeptide in a biological sample derived from the subject, relative to the level present in a reference, wherein an increase in the level of all of said Markers indicates that aggressive therapy is appropriate for the subject, and the absence of an increase in the level of all of said Markers indicates that conventional therapy is appropriate.

21. A method of monitoring neoplasia therapy in a subject, the method comprising determining the level of a Marker selected from the group consisting of OCT3/4, Nanog, Sox2, c-Myc and Klf4 nucleic acid molecule or polypeptide in a biological sample derived from the subject, relative to the level present in a reference, wherein a neoplasia therapy that reduces the level of said marker is identified as effective.

22. (canceled)

23. The method of claim 12, wherein the biological sample is a tissue sample selected from the list consisting of prostate tissue, renal tissue, bladder tissue, breast tissue, skin, and connective tissue.

24. The method of claim 12, wherein the biological sample is a biologic fluid selected from the list consisting of blood, serum, plasma, ejaculate, or urine.

25-28. (canceled)

29. An isolated stem-like cancer-initiating cell having increased expression of two or more of an OCT3/4, Nanog, Sox2, c-Myc and Klf4 nucleic acid molecule or polypeptide.

30-32. (canceled)

33. A pharmaceutical composition for the treatment or prevention of cancer, the composition comprising an agent that reduces the expression or activity of a polypeptide or nucleic acid molecule selected from the group consisting of OCT3/4, Nanog, Sox2, c-Myc and Klf4 nucleic acid molecule or polypeptide.

34-36. (canceled)

37. A method for determining the Marker profile of a neoplasia, the method comprising quantifying the level of two or more Markers selected from the group consisting of OCT3/4, Nanog, Sox2, c-Myc or Klf4 in a biologic sample, wherein the level of Marker in the sample relative to the level in a reference determines the Marker profile of the neoplasia.

38. The method of claim 1, wherein the neoplasia is selected from the group consisting of prostate cancer, renal carcinoma, bladder cancer, breast cancer, melanoma, and sarcoma.

39. A kit for the analysis of OCT3/4, Nanog, Sox2, c-Myc or Klf4, the kit comprising at least one primer or antibody capable of specifically binding or hybridizing to OCT3/4, Nanog, Sox2, c-Myc or Klf4 polypeptide or nucleic acid molecule, and directions for using the primer or antibody for the analysis of OCT3/4, Nanog, Sox2, c-Myc or Klf4.

40-47. (canceled)

48. The method of claim 12, wherein the neoplasia is selected from the group consisting of prostate cancer, renal carcinoma, bladder cancer, breast cancer, melanoma, and sarcoma.

49. The method of claim 20, wherein the neoplasia is selected from the group consisting of prostate cancer, renal carcinoma, bladder cancer, breast cancer, melanoma, and sarcoma.
